SEQUENCE LISTING (68 sequences)

SEQ ID NO: 1 - DNA, 18 bp, synthetic construct
  source 1..18: mol_type = other DNA
cggtggacaaattgtcac

SEQ ID NO: 2 - DNA, 28 bp, synthetic construct
  source 1..28: mol_type = other DNA
ttacaagcttaaagaatgtctgaacact

SEQ ID NO: 3 - DNA, 27 bp, synthetic construct
  source 1..27: mol_type = other DNA
ttgaatttaggtgaaacatttgtcacg

SEQ ID NO: 4 - DNA, 22 bp, synthetic construct
  source 1..22: mol_type = other DNA
cttctctggatttaacacactt

SEQ ID NO: 5 - DNA, 51 bp, synthetic construct
  source 1..51: mol_type = other DNA
tcagcacacaaagccaaaaatttatttttctgtgcaaaggaaattaaggag

SEQ ID NO: 6 - DNA, 49 bp, synthetic construct
  source 1..49: mol_type = other DNA
tattggtggagctaaacttaaagccttttctgtacaatccctttgagtg

SEQ ID NO: 7 - DNA, 59 bp, synthetic construct
  source 1..59: mol_type = other DNA
ttacaagcttaaagaatgtctgaacactaaattacaagcttaaagaatgtctgaacact

SEQ ID NO: 8 - DNA, 57 bp, synthetic construct
  source 1..57: mol_type = other DNA
ttgaatttaggtgaaacatttgtcacgaaattgaatttaggtgaaacatttgtcacg

SEQ ID NO: 9 - DNA, 59 bp, synthetic construct; identical to SEQ ID NO: 7
  source 1..59: mol_type = other DNA
  misc_feature 59: note = 3'-3' phosphodiester bond to a thymidine

SEQ ID NO: 10 - DNA, 57 bp, synthetic construct; identical to SEQ ID NO: 8
  source 1..57: mol_type = other DNA
  misc_feature 57: note = 3'-3' phosphodiester bond to a thymidine

SEQ ID NO: 11 - DNA, 90 bp, synthetic construct
  source 1..90: mol_type = other DNA
  misc_feature 90: note = 3'-3' phosphodiester bond to a thymidine
ttacaagcttaaagaatgtctgaacactaaattacaagcttaaagaatgtctgaacactaaattacaagcttaaagaatgtctgaacact

SEQ ID NO: 12 - DNA, 87 bp, synthetic construct
  source 1..87: mol_type = other DNA
  misc_feature 87: note = 3'-3' phosphodiester bond to a thymidine
ttgaatttaggtgaaacatttgtcacgaaattgaatttaggtgaaacatttgtcacgaaattgaatttaggtgaaacatttgtcacg

SEQ ID NO: 13 - DNA, 22 bp, synthetic construct
  source 1..22: mol_type = other DNA
cctatccgtatggtggataacg

SEQ ID NO: 14 - DNA, 21 bp, synthetic construct
  source 1..21: mol_type = other DNA
gtcggaagctcctatgacaat

SEQ ID NO: 15 - DNA, 45 bp, synthetic construct
  source 1..45: mol_type = other DNA
accggatcgatgtgtactgagattttccaacaagaaggcgtactc

SEQ ID NO: 16 - DNA, 40 bp, synthetic construct
  source 1..40: mol_type = other DNA
atccaaccgtcggtcggattttttgatcgtctcggctagt

SEQ ID NO: 17 - DNA, 18 bp, synthetic construct
  source 1..18: mol_type = other DNA
gatcgagcaagccatctg

SEQ ID NO: 18 - DNA, 19 bp, synthetic construct
  source 1..19: mol_type = other DNA
tgatcagctcggtcttgta

SEQ ID NO: 19 - DNA, 60 bp, synthetic construct
  source 1..60: mol_type = other DNA
gatcgagcaagccatctgaaagatcgagcaagccatctgaaagatcgagcaagccatctg

SEQ ID NO: 20 - DNA, 63 bp, synthetic construct
  source 1..63: mol_type = other DNA
tgatcagctcggtcttgtaaaatgatcagctcggtcttgtaaaatgatcagctcggtcttgta

SEQ ID NO: 21 - DNA, 20 bp, synthetic construct
  source 1..20: mol_type = other DNA
ctttccagagaagctttgct

SEQ ID NO: 22 - DNA, 22 bp, synthetic construct
  source 1..22: mol_type = other DNA
agcaaagatcattagggattat

SEQ ID NO: 23 - DNA, 44 bp, synthetic construct
  source 1..44: mol_type = other DNA
cttgtattactactgccccttcacgatccactttggaaaggacc

SEQ ID NO: 24 - DNA, 48 bp, synthetic construct
  source 1..48: mol_type = other DNA
tgacataaaagtagtgccaagaagattttacaatcatcacctgccatc

SEQ ID NO: 25 - DNA, 20 bp, synthetic construct
  source 1..20: mol_type = other DNA
ggtttattacagggacagca

SEQ ID NO: 26 - DNA, 18 bp, synthetic construct
  source 1..18: mol_type = other DNA
atcctgtctacttgccac

SEQ ID NO: 27 - DNA, 67 bp, synthetic construct
  source 1..67: mol_type = other DNA
ctttccagagaagctttgctaaactttccagagaagctttgctaaactttccagagaagctttgcta

SEQ ID NO: 28 - DNA, 72 bp, synthetic construct
  source 1..72: mol_type = other DNA
agcaaagatcattagggattataaaagcaaagatcattagggattataaaagcaaagatcattagggattat

SEQ ID NO: 29 - DNA, 21 bp, synthetic construct
  source 1..21: mol_type = other DNA
acttgtcttggggaatatctc

SEQ ID NO: 30 - DNA, 18 bp, synthetic construct
  source 1..18: mol_type = other DNA
atgctggagcaaaaagct

SEQ ID NO: 31 - DNA, 44 bp, synthetic construct
  source 1..44: mol_type = other DNA
cgagtcatgattgggccatgacagtgtcatcatttgaaaggttt

SEQ ID NO: 32 - DNA, 46 bp, synthetic construct
  source 1..46: mol_type = other DNA
aaggtgtaacggcagcatgtccgaatttccttttttaactagccat

SEQ ID NO: 33 - DNA, 19 bp, synthetic construct
  source 1..19: mol_type = other DNA
gctaagagagcaattgagc

SEQ ID NO: 34 - DNA, 19 bp, synthetic construct
  source 1..19: mol_type = other DNA
atgtaggatttgctgagct

SEQ ID NO: 35 - DNA, 69 bp, synthetic construct
  source 1..69: mol_type = other DNA
acttgtcttggggaatatctcaaaacttgtcttggggaatatctcaaaacttgtcttggggaatatctc

SEQ ID NO: 36 - DNA, 60 bp, synthetic construct
  source 1..60: mol_type = other DNA
atgctggagcaaaaagctaaaatgctggagcaaaaagctaaaatgctggagcaaaaagct

SEQ ID NO: 37 - DNA, 22 bp, synthetic construct
  source 1..22: mol_type = other DNA
ttggaatgctcactttactgaa

SEQ ID NO: 38 - DNA, 18 bp, synthetic construct
  source 1..18: mol_type = other DNA
cgatatcgcgcgtctgtc

SEQ ID NO: 39 - DNA, 41 bp, synthetic construct
  source 1..41: mol_type = other DNA
tgcttggttcgcccgtttaagatggcgtagttgtgctctgg

SEQ ID NO: 40 - DNA, 42 bp, synthetic construct
  source 1..42: mol_type = other DNA
ttcccaatagcttgatcgccctcccacttaaagacttggtgt

SEQ ID NO: 41 - DNA, 20 bp, synthetic construct
  source 1..20: mol_type = other DNA
attggtaaatccttgctgct

SEQ ID NO: 42 - DNA, 19 bp, synthetic construct
  source 1..19: mol_type = other DNA
tgggcgtggttaaggatga

SEQ ID NO: 43 - DNA, 66 bp, synthetic construct
  source 1..66: mol_type = other DNA
attggtaaatccttgctgctaaaattggtaaatccttgctgctaaaattggtaaatccttgctgct

SEQ ID NO: 44 - DNA, 63 bp, synthetic construct
  source 1..63: mol_type = other DNA
tgggcgtggttaaggatgaaaatgggcgtggttaaggatgaaaatgggcgtggttaaggatga

SEQ ID NO: 45 - DNA, 150 bp, synthetic construct
  source 1..150: mol_type = other DNA
acctccatggtcctcgacgcgatcgagcaagccatctggacccgccaacaagaaggcgtactcgacctgaaagacgttatccaccatacggataggggatctcagtacacatcgatccggttcagcgagcggctcgccgaggcaggcatc

SEQ ID NO: 46 - DNA, 150 bp, synthetic construct
  source 1..150: mol_type = other DNA
ccgccagggcttgccgggtttgatcagctcggtcttgtataggccgttgatcgtctcggctagtgcattgtcataggagcttccgaccgctccgaccgacggttggatgcctgcctcggcgagccgctcgctgaaccggatcgatgtgta

SEQ ID NO: 47 - DNA, 46 bp, synthetic construct
  source 1..46: mol_type = other DNA
atcgattaatacgactcactatagggacctccatggtcctcgacgc

SEQ ID NO: 48 - DNA, 39 bp, synthetic construct
  source 1..39: mol_type = other DNA
tacgttaattaaccctcactaaagccgccagggcttgcc

SEQ ID NO: 49 - DNA, 120 bp, synthetic construct
  source 1..120: mol_type = other DNA
acaaaaattcaaaattttcgggtttattacagggacagcagaaatccactttggaaaggaccagcaaagctcctctggaaaggtgaaggggcagtagtaatacaagataatagtgacata

SEQ ID NO: 50 - DNA, 120 bp, synthetic construct
  source 1..120: mol_type = other DNA
ctcatcctgtctacttgccacacaatcatcacctgccatctgttttccataatccctaatgatctttgcttttcttcttggcactacttttatgtcactattatcttgtattactactgc

SEQ ID NO: 51 - DNA, 55 bp, synthetic construct
  source 1..55: mol_type = other DNA
atcgattaatacgactcactatagggacaaaaattcaaaattttcgggtttatta

SEQ ID NO: 52 - DNA, 44 bp, synthetic construct
  source 1..44: mol_type = other DNA
tacgttaattaaccctcactaaagctcatcctgtctacttgcca

SEQ ID NO: 53 - DNA, 140 bp, synthetic construct
  source 1..140: mol_type = other DNA
gatttcatcgattatgaggagctaagagagcaattgagctcagtgtcatcatttgaaaggtttgagatattccccaagacaagttcatggcccaatcatgactcgaacaaaggtgtaacggcagcatgtcctcatgctgg

SEQ ID NO: 54 - DNA, 142 bp, synthetic construct
  source 1..142: mol_type = other DNA
gacttctttccctttatcattaatgtaggatttgctgagctttgggtatgaatttccttttttaactagccatattaaatttttgtagaagctttttgctccagcatgaggacatgctgccgttacacctttgttcgagtca

SEQ ID NO: 55 - DNA, 46 bp, synthetic construct
  source 1..46: mol_type = other DNA
atcgattaatacgactcactataggggatttcatcgattatgagga

SEQ ID NO: 56 - DNA, 44 bp, synthetic construct
  source 1..44: mol_type = other DNA
tacgttaattaaccctcactaaaggacttctttccctttatcat

SEQ ID NO: 57 - DNA, 151 bp, synthetic construct
  source 1..151: mol_type = other DNA
acctccatggtcctcgacgcttggaatgctcactttactgaacataaatcacagggcgtagttgtgctctggaatgagaataagcagcaaggatttaccaataatcttaaacgggcgaaccaagcatttttacccgcatctacctttaaaa

SEQ ID NO: 58 - DNA, 150 bp, synthetic construct
  source 1..150: mol_type = other DNA
atgatcgcgattccaagtggcgatatcgcgcgtctgtccatcccacttaaagacttggtgttcatccttaaccacgcccaaatcgagggcgatcaagctattgggaattttaaaggtagatgcgggtaaaaatgcttggttcgcccgttt

SEQ ID NO: 59 - DNA, 49 bp, synthetic construct
  source 1..49: mol_type = other DNA
tacgttaattaaccctcactaaagatgatcgcgattccaagtggcgata

SEQ ID NO: 60 - DNA, 20 bp, synthetic construct
  source 1..20: mol_type = other DNA
gaccccaaaatcagcgaaat

SEQ ID NO: 61 - DNA, 24 bp, synthetic construct
  source 1..24: mol_type = other DNA
tctggttactgccagttgaatctg

SEQ ID NO: 62 - DNA, 24 bp, synthetic construct
  source 1..24: mol_type = other DNA
accccgcattacgtttggtggacc

SEQ ID NO: 63 - DNA, 26 bp, synthetic construct
  source 1..26: mol_type = other DNA
atattctaagcacacgcctattatag

SEQ ID NO: 64 - DNA, 23 bp, synthetic construct
  source 1..23: mol_type = other DNA
ctaccaatggttctaaagccgaa

SEQ ID NO: 65 - DNA, 24 bp, synthetic construct
  source 1..24: mol_type = other DNA
gagccagaagatctccctcagggt

SEQ ID NO: 66 - DNA, 19 bp, synthetic construct
  source 1..19: mol_type = other DNA
agatttggacctgcgagcg

SEQ ID NO: 67 - DNA, 20 bp, synthetic construct
  source 1..20: mol_type = other DNA
gagcggctgtctccacaagt

SEQ ID NO: 68 - DNA, 23 bp, synthetic construct
  source 1..23: mol_type = other DNA
ttctgacctgaaggctctgcgcg

(19) United States
(12) Patent Application Publication
(10) Pub. No.: US 2025/0171835 A1
(43) Pub. Date: May 29, 2025

(54) DETECTION OF CONDENSED AMPLIFICATION PRODUCTS

(71) Applicants: Vicente Jose PELECHANO GARCIA, Solna (SE); Donal BARRETT, Stockholm (SE); The Board of Trustees of the Leland Stanford Junior University, Stanford, CA (US); RUTGERS, THE STATE UNIVERSITY OF NEW JERSEY, New Brunswick, NJ (US); YALE UNIVERSITY, New Haven, CT (US)

(72) Inventors: Vicente Jose PELECHANO GARCIA, Solna (SE); Donal BARRETT, Stockholm (SE); Lars M. STEINMETZ, Stanford, CA (US); Mehdi JAVANMARD, West Windsor, NJ (US); Muhammad TAYYAB, New Brunswick, NJ (US); Curt SCHARFE, New Haven, CT (US); Peter B. GRIFFIN, Stanford, CA (US)

(21) Appl. No.: 18/728,823

(22) PCT Filed: Jan. 19, 2023
(86) PCT No.: PCT/US2023/060940

§ 371 Date: Jul. 12, 2024

Related U.S. Application Data
(60) Provisional application No. 63/301,945, filed on Jan. 21, 2022.

(51) Int. Cl. C12Q 1/6844; B01L 3/00; C12Q 1/70

(52) U.S. Cl. CPC C12Q 1/6844; B01L 3/502715; C12Q 1/701; B01L 2200/0647; B01L 2200/16; B01L 2300/0645; C12Q 2600/156

(57) ABSTRACT

Provided herein is a method for detecting a target nucleic acid in a sample. In some embodiments, the method may comprise: (a) amplifying the target nucleic acid isothermally in the presence of one or more compaction oligonucleotides to produce a product that comprises condensed amplification products; (b) flowing the product through a microfluidic channel; and (c) detecting a change in impedance as the condensed amplification products pass through the microfluidic channel. A microfluidic system and kit for preforming the method are also provided.

### CROSS-REFERENCING

This application is a § 371 national phase of International Application No. PCT/US2023/060940, filed on Jan. 19, 2023, which claims the benefit of U.S. provisional application Ser. No. 63/301,945, filed on Jan. 21, 2022, which application are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML entitled “STAN-1927_SeqList_01-02-2025”. This file was created on Jan. 2, 2025, and has a size of 64,658 bytes. The contents of the Sequence Listing XML are incorporated herein by reference in their entirety.

### BACKGROUND

Fast and accurate detection of genetic material is a cornerstone for diagnostics with far reaching applications, including the identification of infectious diseases and patient biomarkers. This necessity for sensitive and scalable detection of pathogens has been clearly highlighted during the recent SARS-COV-2 pandemic. This has led to a resurgence of existing methods and the development of novel diagnostics methods targeting viral nucleic acids or proteins. The use of protein based diagnostic approaches have been a major tool in the COVID pandemic. Due to their intrinsic simplicity and scalability, these approaches have been key to minimize the spread of disease, with a clear example being the use of rapid antigen detection coupled with lateral flow detection. However, protein-based tests often require the development of high-quality antibodies.

Alternatively, nucleic-acid based approaches are easier and quicker to develop, intrinsically flexible and thus a first line of defense against emerging pathogens. RT-qPCR was successfully used to detect virus infection even before SARS-COV-2 was declared a pandemic. Additionally, nucleic acid based approaches are able to amplify the underlying signal and thus possess in general a higher sensitivity than protein based approaches. To complement RT-qPCR many other nucleic acid-based approaches have been developed, including loop mediated isothermal amplification (LAMP).

The LAMP reaction provides a simple, portable form of rapid nucleic acid amplification. With the aid of 4-6 target specific primers together with a strand displacing polymerase the reaction which does not require a thermocycler provides a low-cost sensitive alternative to the standard PCR. LAMP combined with a reverse transcriptase (RT-LAMP) has been used to detect several pathogens previously. The readouts for such methods are generally fluorescent and colorimetric. However, fluorescence-based methods have a high reagent cost and require a specialized readout system. Colorimetric methods, on the other hand, can be read by eye but, under certain circumstances, are susceptible to false positives because, for example, raw saliva can be acidic and can require additional steps prior to testing to neutralize such samples.

There is therefore an ongoing need for new diagnostic platforms, particularly for detecting pathogens such as viruses.

### SUMMARY

Provided herein is a method for detecting a target nucleic acid in a sample. In some embodiments, the method may comprise: (a) amplifying the target nucleic acid isothermally in the presence of one or more compaction oligonucleotides to produce a product that comprises condensed amplification products; (b) flowing the product through a microfluidic channel; and (c) detecting a change in impedance as the condensed amplification products pass through the microfluidic channel.

A microfluidic system is also provided. In some embodiments, the microfluidic system may comprise: (i) a reaction chamber comprising reagents for amplifying a target nucleic acid isothermally as well as compaction oligonucleotides, for the production of a product that comprises condensed amplification products; (ii) a microfluidic channel comprising electrodes; and (iii) an impedance detector, wherein the impedance detector is connected to the electrodes and detects the condensed amplification products passing by the electrodes.

A kit is also provided. The method, system and kit have a variety of diagnostic uses.

### BRIEF DESCRIPTION OF THE FIGURES

The skilled artisan will understand that the drawings, described below, are for illustration purposes only. The drawings are not intended to limit the scope of the present teachings in any way.

FIGS. 1A-1E: Electrical detection of DNA Nanoballs. (FIG. 1A) Formation of DNA nanoball using compaction oligos. (FIG. 1B) Fluorescence image of DNA nanoballs (FIG. 1C) Fluorescent image of 1 uM MyOne Dynabeads as size reference. (FIG. 1D) Passive flow of DNA nanoballs in a microfluidic chip made of PDMS on a glass substrate integrated with gold electrodes. The passage of DNA nanoballs through the gold electrodes occludes the current path and disturbs the electric field formed between the gold electrodes. (FIG. 1E) A schematic illustrating the electronic readout system used for the microfluidic chip with integrated gold electrodes.

FIGS. 2A-2E: Microfluidic chip for impedance based detection of DNA nanoballs. (FIG. 2A) A picture of the microfluidic chip (FIG. 2B) Microscopic image of the channel with the integrated gold electrodes. (FIGS. 2C-2E) Principle of detection of DNA nanoballs. The passing of a DNA nanoball through the integrated gold electrodes produces a spike signature in the impedance response of the system. This impedance response is recorded as a single DNA nanoball.

FIGS. 3A-3F: Optimization, Limit of Detection, and Assay time (FIG. 3A) Bar graph for nanoballs detected in 1:1 Compaction Oligos vs. 9:1 Compaction Oligos with DI water as Negative Control (FIG. 3B) Boxplot for confirming first round of optimizations. The error bars represent a 95% Confidence Interval (CI) (FIG. 3C) Bright field microscopic image of nanoballs with 9:1 Compaction oligos. (FIG. 3D) Dilution series experiment to determine the Limit of Detection using two compaction oligos and 9:1 ratio. This experiment is allowed to run for 10 minutes. (FIG. 3E) Boxplot for the dilution series experiment. The error bars represent 95% CI. (FIG. 3F) Nanoballs detected in different assay times. The error bars represent a 95% CI.

FIGS. 4A-4D: Optimization of experimental protocol for the detection of DNA nanoballs (FIG. 4A) 2 repeat compaction oligos (FIG. 4B) 3 repeat compaction oligos (FIG. 4C) ssDNA binding protein with 2 repeat compaction oligos (FIG. 4D).

FIGS. 5A and 5B: Testing Clinical samples for COVID-19 patients (FIG. 5A) Nanoballs detected from heat inactivated surplus aliquots of SARS-COV-2 positive and negative nasopharyngeal samples that had previously been clinically diagnosed for COVID-19 by RT-PCR. The samples were divided into 4 groups: 5 SARS-COV-2 negative patient samples (NC2, yellow), 15 SARS-COV-2 positive samples divided into 3 sub groups based on CT Results as follows: Low CT 17-20=Hi Conc (Red), Medium CT 22-24=Med Conc (Green), High CT 26-27=Low Conc (Blue), and 1 Water Control (NC1, purple) (FIG. 5B) Boxplot summarizing the categorical data. The error bars represent the Standard Deviation for each group.

FIGS. 6A-6E: Testing Multiple Pathogens (FIG. 6A) HIV (FIG. 6B) Influenza (FIG. 6C) Mycobacterium (FIG. 6D) β-Lactamase (FIG. 6E) Boxplot summarizing the results for the multiple pathogens. The error bars represent 95% CI.

FIG. 7 illustrates a microfluidic cartridge capable of isothermal amplification and impedance detection.

FIG. 8 Depicting typical a LAMP reaction (panel A) and production of numerous long dumbbell like structures, subsequently compaction oligos hybridize (panel B) via compaction oligo with 2 repeats or alternatively (panel C) compaction oligo with 3 repeats. Further in (panel D) compaction using a compaction oligo with 2 repeats targeting an amplified region (N) not contained in the sequences of the standard LAMP primers used for isothermal amplification. LAMP primers targeting six target regions consisting of three Forward (F1, F2, F3) and three Backwards (B1, B2, B3) regions as per typical LAMP reactions (Rabe et al Proc Natl Acad Sci USA. 2020 Sep. 29 117:24450-8). Complementary sequence denoted by c, L denotes Loop.

FIG. 9 shows various configurations table for electrical parameter optimization.

FIGS. 10A-10D show the results of various optimization parameters.

### DESCRIPTION

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N. Y. (1991) provide one of ordinary skill in the art with the general meaning of many of the terms used herein. Still, certain terms are defined below for the sake of clarity and ease of reference.

Other definitions of terms may appear throughout the specification.

As noted above, this disclosure provides a method for detecting a target nucleic acid in a sample. In some embodiments, the method may comprise: (a) amplifying the target nucleic acid isothermally in the presence of one or more compaction oligonucleotides to produce a product that comprises condensed amplification products; (b) flowing the product through a microfluidic channel; and (c) detecting a change in impedance as the condensed amplification products pass through the microfluidic channel.

One advantage of impedance detection is that electrical signals are straightforward to detect, making an inexpensive, portable device possible. It also avoids the more complex fluorescent detection methods conventionally used.

In some embodiments, the amplifying may be done by loop-mediated isothermal amplification (LAMP) or reverse transcription loop-mediated isothermal amplification (RT-LAMP). In LAMP methods, the target sequence may be amplified at a constant temperature of 60-65° C. (140-149° F.) using either two or three sets of primers and a polymerase with high strand displacement activity in addition to a replication activity. Typically, four different primers are used to amplify a distinct region in the target nucleic acid, which increases specificity. An additional pair of “loop primers” can further accelerate the reaction. See, e.g., Notomi et al (Nucleic Acids Res. 2000 28: 63e-63), U.S. Pat. No. 6,410,278 and Nagamine et al (Mol. Cell. Probes 2002 16:223-9), which are incorporated by reference herein.

Alternatively, the amplifying may be done by rolling circle amplification (RCA), which is an isothermal amplification that generates linear concatemerized copies of a circular nucleic acid template using a strand-displacing polymerase. RCA is well known in the molecular biology arts and is described in a variety of publications including, but not limited to Lizardi et al (Nat. Genet. 1998 19:225-232), Schweitzer et al (Proc. Natl. Acad. Sci. 2000 97:10113-10119), Wiltshire et al (Clin. Chem. 2000 46:1990-1993) and Schweitzer et al (Curr. Opin. Biotech 2001 12:21-27), which are incorporated by reference herein.

In any embodiment, the amplification reagents may contain a thermolabile uracil-DNA-glycosylase and dUTP, which provides a way to decontaminate carry-over amplification products from one reaction to the next (see, e.g., Zeng et al Analyst 2020 145:7048-7055).

In any embodiment, the sample may be a clinical sample obtained from a patient, particularly a liquid sample such as a e.g., a nasal swab, blood plasma, saliva, urine, amniotic fluid, aqueous humor, vitreous humor, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, or urine.

In particular embodiments, the primers may be directed to a target nucleic acid in an infectious agent (i.e., a pathogen), e.g., a virus, bacteria, or another organism. The target nucleic acid may be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus, West Nile virus and Ebola virus. The present invention is not, however, limited to the detection of nucleic acid, e.g., DNA or RNA, sequences from the aforementioned viruses, but can be applied without any problem to other pathogens important in veterinary and/or human medicine. Other pathogens that may be detected in using the present method include, but are not limited to: Varicella zoster; Staphylococcus epidermidis, Escherichia coli, methicillin-resistant Staphylococcus aureus (MSRA), Staphylococcus aureus, Staphylococcus hominis, Enterococcus faecalis, Pseudomonas aeruginosa, Staphylococcus capitis, Staphylococcus warneri, Klebsiella pneumoniae, Haemophilus influenzae, Staphylococcus simulans, Streptococcus pneumoniae and Candida albicans; gonorrhea (Neisseria gorrhoeae), syphilis (Treponena pallidum), clamydia (Clamyda tracomitis), nongonococcal urethritis (Ureaplasm urealyticum), chancroid (Haemophilus ducreyi), trichomoniasis (Trichomonas vaginalis); Pseudomonas aeruginosa, methicillin-resistant Staphylococcus aureus (MSRA), Klebsiella pneumoniae, Haemophilis influenzae, Staphylococcus aureus, Stenotrophomonas maltophilia, Haemophilis parainfluenzae, Escherichia coli, Enterococcus faecalis, Serratia marcescens, Haemophilis parahaemolyticus, Enterococcus cloacae, Candida albicans, Moraxiella catarrhalis, Streptococcus pneumoniae, Citrobacter freundii, Enterococcus faecium, Klebsella oxytoca, Pseudomonas fluorscens, Neiseria meningitidis, Streptococcus pyogenes, Pneumocystis carinii, Klebsella pneumoniae Legionella pneumophila, Mycoplasma pneumoniae, and Mycobacterium tuberculosis, etc.

In some embodiments, the primers may be designed to amplify a target nucleic acid from a coronavirus or influenza virus. For example, the target sequence could be in the SARS-COV-2 genome. For the detection of SARS-COV-2, LAMP primers may be designed using the same strategies as outlined in Huang et al (Microb. Biotechnol. 2020 13:950-961), Wang et al (Biosens. Bioelectron. 2021 172:112766), Dao et al (Sci Transl Med. 2020 12 (556): eabc7075) and Schermer et al (PLOS One. 2020 15: e0238612), although other sequences could be used.

As noted above, the method uses “compaction oligonucleotides”. Isothermal amplification such as RCA and LAMP result in a product (i.e., a strand) that has a repeated sequence copied from the target nucleic acid. Compaction oligonucleotides comprise sequences that hybridize to different repeats in the amplification product. When a compaction oligonucleotide hybridizes to an amplification product, the product condenses into a compact form that, in some cases, can be in the range of 0.1-2 microns in diameter. These products, which are composed of an amplification product that is condensed by base-pairing to multiple compaction oligonucleotides, is referred to as a “condensed amplification product’ (or “DNA nanoball”) herein.

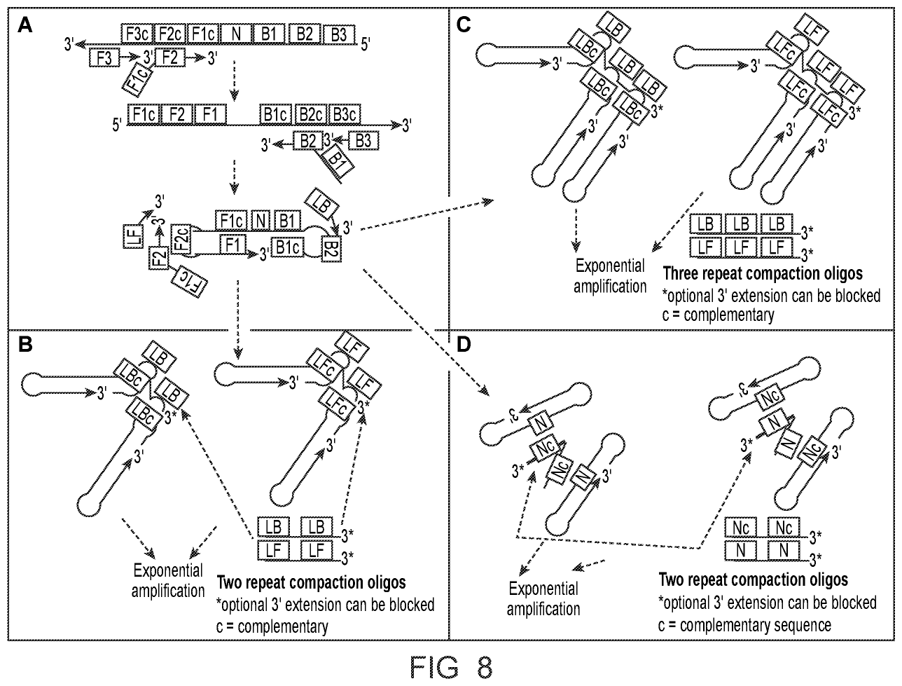

Strategies for the design of compaction oligonucleotides are described in Clausson et al (Sci Rep 2015 5:12317) and shown in FIG. 8. In general, a compaction oligonucleotide comprises a first sequence of 10-30 nucleotides that hybridizes to a first repeat in the product and a second sequence of 10-30 nucleotides that hybridizes to a second repeat in the product. In some embodiments, the first and second sequences can be the same. In LAMP, some of the sequence in the LAMP product is contributed by a primer that is commonly known as a “loop primer”. As such, in some embodiments, the compaction oligonucleotide may have two copies of a sequence in a loop primer, or its complement, where the copies are 10-30 nucleotides in length and hybridize to the product. Compaction oligonucleotides can be blocked at the 3′ end to avoid extension and degradation, e.g., using a 2′O-methyl group. In some embodiments, a compaction oligonucleotide may be designed to hybridize to a sequences that are part of the amplified template (i.e., not a primer sequence), as shown in FIG. 8.

Impedance cytometry is then used to detect the condensed amplification products (the “DNA nanoballs”). When a condensed amplification product flows through the sensing region, it partially impedes the AC electric field generated between the two electrodes, which results in an instantaneous frequency-dependent drop in ionic current, i.e., a momentary increase in impedance. In some embodiments, one can use a multi-frequency lock-in amplifier to measure impedance at different frequencies, e.g., at multiple different frequencies ranging from 100 kHz to 20 MHz. For example, one electrode can be excited with a combination of multiple different frequency AC signals and the other electrode can be tied to a transimpedance amplifier. The general design of the detection system can be adapted from the systems described in Sui et al (Sci Rep. 2021 11:6490) and Petchakup et al (Micromachines (Basel). 2017 8:87), for example. In any embodiment, the microfluidic channel through which the condensed amplification products pass may have a width of 10-200 microns and, as would be apparent, the microfluidic channel may have two or more electrodes, and wherein the change in impedance is measured when the condensed amplification products pass by the electrodes. The electrodes may have a width in the range of 1-50 microns and may be spaced apart by 1-50 microns. In any embodiment, the electrodes provide an alternating current which may have a frequency of below 100 kHz (e.g., in range of 10-50 kHz) and a voltage in the range of 100-1000 mv.

In any embodiment, the method can comprise amplifying a second target nucleic acid isothermally in the presence of one or more compaction oligonucleotides to produce second condensed amplification products, wherein the second condensed amplification products can be distinguished from the first products by their impedance profile. In these embodiments, one or more of the oligonucleotides used for amplification may be conjugated to a binding moiety (e.g., biotin, click groups, etc.) such that the condensed amplification products can be bound to different detection agents (e.g., gold nanoparticles, proteins), etc., which will allow one to distinguish between the different products by their impedance. Specifically, the method can be used to detect different condensed amplification products by their coating or other modification, e.g., metal (e.g., gold) nanoparticles, proteins, polymers, polysaccharides, proteins, or modified DNA/RNA residues, etc. As one can direct the association of those “modifiers” to DNA nanoballs generated from specific target regions (e.g., by using modified primers), one can envision the simultaneous generation of several DNA nanoballs with different properties. In one example, some primers may target SARS-CoV-2 and other primers that have a biotin group may target influenza virus. In use, the influenza products can be bound to gold nanoparticles, allowing those targets to be distinguished by their impedance. This allows the method to be multiplexed.

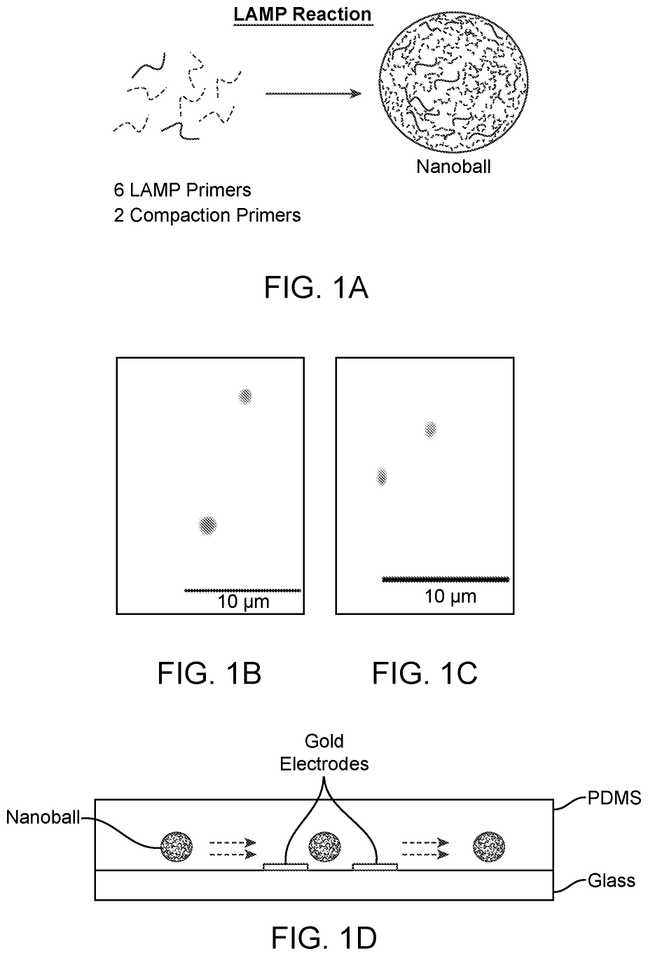

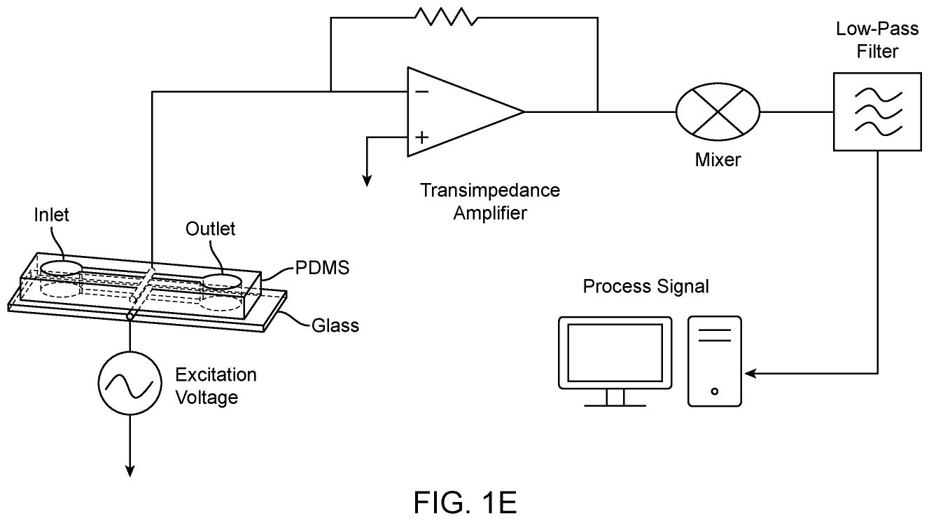

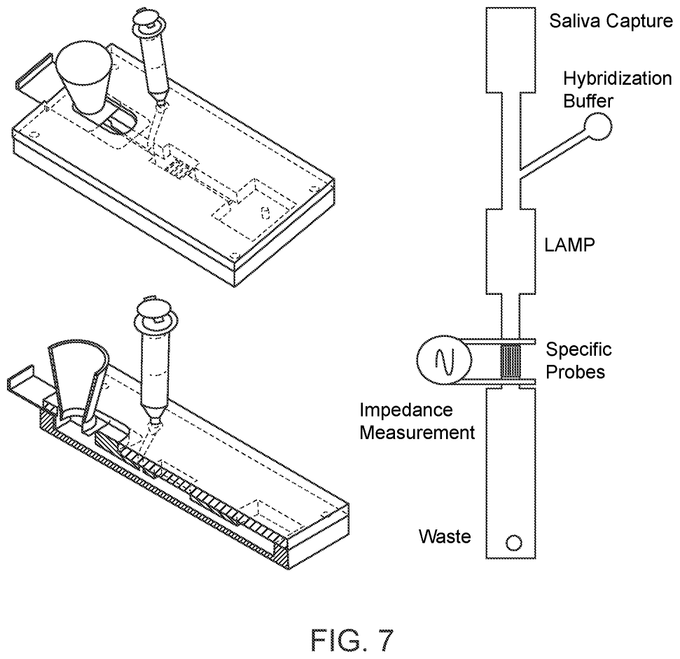

Also provided is a microfluidic system which, in some embodiments, may comprise: (i) a reaction chamber comprising reagents for amplifying target nucleic acid isothermally and compaction oligonucleotides, for the production of a product that comprises condensed amplification products; (ii) a microfluidic channel comprising electrodes; and (iii) an impedance detector, wherein the impedance detector is connected to the electrodes and detects the condensed amplification products passing by the electrodes. The reaction chamber may comprise reagents for amplifying the target nucleic acid by LAMP. Some of the components of this device are illustrated in FIG. 1E and FIG. 7. The components of the device of FIG. 1E are described below. The device of FIG. 7 has a saliva collection funnel, a LAMP amplification chamber an a waste chamber (having a volume of 10 ul to 1 ml, for example). In use, a user would open the cap on the hybridization buffer, would provide a gravity-driven flow of fluid to move the condensed products through the detection chamber and impedance sensor. Other implementations are possible. For example, the device could use blood.

Also provided is a kit comprising: reagents for amplifying target nucleic acid isothermally (e.g., LAMP, RT-LAMP or RCA primers, dNTPs, polymerase etc.), compaction oligonucleotides for condensing the amplification products, compaction oligonucleotides, and a microfluidic system comprising (i) a reaction chamber that may contain those reagents, (ii) a microfluidic channel comprising electrodes; and (iii) an impedance detector, wherein the impedance detector is connected to the electrodes and detects the condensed amplification products passing by the electrodes. Depending on which agent is going to be tested, the kit may further comprise other elements such as, e.g., a nasal swab, saliva collection tube or a lance.

In any embodiment, the method may be run at the site at which the sample has been obtained, thereby providing an instant result. In some embodiments, the method may comprise providing a report indicating whether the subject is positive for a particular pathogen based on the result of the method. In some embodiments, the report e.g., in an electronic form, may be forwarded to a doctor or other medical professional to help identify a suitable course of action, e.g., to identify a suitable therapy for the subject. The report may be used along with other metrics as a diagnostic to determine whether the subject has an infection.

In any embodiment, the report can be forwarded to a “remote location”, where “remote location,” means a location other than the location at which sample is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the report may be analyzed by an MD or other qualified medical professional, and a report based on the results of the analysis of the obtained data may be forwarded to the subject from which the sample was obtained.

In order to further illustrate the present invention, the following specific examples are given with the understanding that they are being offered to illustrate the present invention and should not be construed in any way as limiting its scope.

### Experimental

Fast and accurate molecular diagnostic techniques are a cornerstone of many industries. Traditional methods of detection often require extensive sample preparation steps, equipment and or reagents which limit applicability, particularly in resource limited resource setting. Here new method of label free impedance-based detection of self-assembling DNA nanoballs simply by adding two compaction oligos to a standard isothermal amplification reaction (in this case, RT-LAMP). The impedance-based detection provides quantized reads of DNA nanoballs from various sequences initially with as little as 10 copies of synthetic SARS-COV-2 RNA and further validated with clinical COVID-19 patient samples. Detection of multiple DNA and RNA pathogenic sequences from various viral and bacterial sources has been demonstrated, which paves the way for a novel and simple impedance-based multi-pathogen POC nucleic acid detection system.

### Methods and Materials

### RT-LAMP Primers, Compaction Oligonucleotide Design and Synthetic RNA/DNA Positive Controls

Asle primers designed against the orflab region of SARS-Cov-2 viral genome were modified to make “compaction oligos”. For two of the six standard Asle primers, namely As1 LF and As1 LB (see Tables 1-3 below), the oligo sequence was duplicated, placing a 3 nucleotide AAA linker sequence between each to form a ‘compaction’ oligo for each. Two such repeat sequences with one spacer (so called two repeat compaction oligos) were included for the first compaction oligo designs and optimization experiments as well as modifying with or without a 3 prime Inverted dT nucleotide modification. In later optimisations described below a further repeat (three repeat compaction oligos) of the original sequence and a second AAA spacer (see Tables 1-3 below) was added. To track potential Nanoball formation with Fluorescence microscopy a 5′ fluorophore modification:

5′ 6 FAM (Fluorescein) was added to the standard As1 F3 primer. Established RT-LAMP primers were also modified to produce three repeat compaction oligos against Mycobacterium tuberculosis (39), HIV (40), Influenza-A/H1N1 (41), and a common B-lactamase based antimicrobial resistance gene (42). All oligonucleotides were purchased from IDT (Integrated DNA Technologies, 1710 Commercial Park Coralville, IA 52241 USA) with standard desalting and dissolved in nuclease free water upon arrival. Various versions of compaction oligos were investigated including 3′ modifications (invdT) to prevent oligo extension and polymerisation in attempts to favor compaction. The first compaction oligo designs included so-called ‘two repeat’ compaction oligos.

Synthetic fragments of SARS-COV-2-RNA were generated as previously described (19) by in-vitro transcription of PCR fragments with sequences including a T7 promoter and a part of SARS-COV-2 sequence targeted by Asle primers. The PCR product was amplified using T7-HMS1-FW (TAATACGACTCACTATAGGGTGCTTGTG AAATTGTCGGTGGA) and HMS1_rv (GCTTTTAGAGGCATGAGTAGGC). The synthetic RNA was produced using TranscriptAid T7 High Yield Transcription Kit (Thermo Fisher Scientific, Waltham, MA, USA), then DNase treated with TURBO DNA-free kit (Thermo Fischer Scientific, Waltham, MA, USA) and subsequently purified with Ampure XP beads (Beckman Coulter, Brea, CA, USA). RNA was quantified using Qubit RNA HS Assay kit (Thermo Fisher Scientific, Waltham, MA, USA).

To generate synthetic targets for the four non-SARS-COV-2 gene targets mentioned above pairs of single stranded DNA oligonucleotide ultramers were ordered from IDT (Integrated DNA Technologies, Coralville, IA, USA) coding for regions targeted by their respective RT-LAMP primers. Each ultramer in a pair coded for one of the two strands of a target region with a short overlapping section in each 3 prime end which facilitated subsequent annealing and Taq polymerase extension to produce a full length double strand DNA fragment. These dsDNA ultramers were further amplified with target specific PCR primers (see Tables 1-3 below) which contained either a T7 or T3 promoter sequence in the 5-prime region. ssRNA was produced from these targets as previously described via the TranscriptAid T7 High Yield Transcription Kit (Thermo Fisher Scientific, Waltham, MA, USA). Ultramers were produced via standard desalting and dissolved in nuclease free water upon arrival.

### RT-LAMP

RT-LAMP reactions were assembled in 20 ul reactions on ice in PCR multistrips (Sarstedt, Numbrecht, Germany). Reactions consisted of 10 ul of WarmStart Colorimetric LAMP 2× Master mix with UDG (New England Biolabs, Ipswich, MA, USA), 1 ul of sample (synthetic RNA/DNA or nuclease free water for negative controls) 7 ul nuclease free water and 2 ul 10×LAMP primer mix. The standard primer mix consisted of 2 uM F3-5′-6-FAM, 2 uM B3, 16 uM FIP, 16 uM BIP, 4 uM LF and 4 uM LB. For compaction oligo optimization trials, a 1:1 ratio between standard LF or LB to compaction oligos (2 uM LF Compaction: 2 uM LF and 2 uM LB Compaction oligo: 2 uM LB) or a 9:1 ratio (3.6 uM LF compaction oligo: 0.4 uM LF and 3.6 uM LB compaction oligo: 0.4 uM LB) was used. All conditions were carried out in triplicates.

For optimization tests with ssDNA binding protein EcoSSB, (Promega Corporation, Madison, WI, USA), following RT-LAMP reactions, 0.5 ul of EcoSSB and 2 ul NEB Buffer 4 (New England Biolabs, Ipswich, MA, USA) were added to each reaction and incubated at 37° C. for 30 min.

For optimization tests with biotinylated RT-LAMP primers, 3,3% biotinylated FIP and BIP (0.4 uM of 16 uM of each) were incorporated, and 100,000 20 nM gold coated streptavidin beads (53134-1ML, Darmstadt, Merck) were subsequently added, to each sample.

### Statistical Analysis

A statistical analysis of these data was performed using R software. It was first determined whether the 10 copies of RNA and the negative control can be differentiated in a detection time of 10 minutes. The mean number of peaks detected for the negative control is 23.6 with a standard deviation of 8.1 whereas the mean number of peaks detected for the 10 copies of RNA are 86.3 with a standard deviation of 21.5. Ideally, there should be no peaks in the data from the negative control, however since the system was tuned to be sensitive to minute impedance changes some peaks are observed. These peaks may be due to stray particles in the solution with sizes comparable to that of nanoballs. A Shapiro Wilk normality test was then performed for these distributions to assess the normality of the distributions. The SW normality test yields (W=0.84799, p-value=0.2351) for the negative control and yields (W=0.99982, p-value=0.9744) for the group with 10 copies of RNA indicating that the data does not deviate significantly from normality. A Welch's two sample t-test was performed with the alternative hypothesis that the mean of the control group is less than the group with 10 copies of RNA. The t-test yields (t=−4.7207, df=2.5623, p-value=0.01271) therefore the null hypothesis is rejected and it is concluded that the control group has a statistically significant less mean than the distribution with 10 copies of RNA for 10 minutes. The statistical analysis was similarly performed for ascertaining the statistical difference between the two groups with an assay time of 30 s. The mean and standard deviation for the control group at 30 s is 0.33 and 0.57 respectively; whereas the mean and standard deviation for the group with 10 copies of RNA is 5.33 and 3.51 respectively. Shapiro Wilk tests for control group (W=0.75, p-value<2.2e-16) and for the group with 10 copies of RNA (W=0.99324, p-value=0.8428) demonstrated that the negative control group deviates significantly from normality. Therefore, a non-parametric test such as the Mann Whitney U test is more appropriate in this case. The Mann Whitney test yields a p-value of 0.03826 for an alternative hypothesis that the control group has a mean less than that of the RNA group with 10 copies. Hence the null hypothesis is rejected and it is concluded that the mean number of nanoballs detected in the control group is less than that detected in the group with 10 copies of RNA at 30 s.

### Fluorescent Microscopy Imaging

To verify the production of DNA Nanoballs from the modified RT-LAMP reaction the products were subsequently imaged on a Nikon ECLIPSE Ti inverted research microscope using Plan Apo I 100× oil Ph3 DM (1.45 NA). 1-2 ul per sample was pipetted onto a glass slide, allowed to air dry and a cover slip applied. 5-6-FAM (Fluorescein) fluorescence-based images were captured using excitation and emission filters for GFP. Additionally 1 ul of the 1 uM Dynabeads™ MyOne™ Streptavidin T1 (Thermo Fisher Scientific, Waltham, MA, USA) was imaged as a size reference.

### Microfluidic Chip

The microfluidic chip is made of PDMS on a glass surface with integrated gold electrodes. The first step for the formation of the microfluidic chip is patterning and fabricating the electrodes on the glass wafer. Electrodes are fabricated on glass using standard photolithography on a 3″ fused silica wafer. The process consists of photo-patterning resist on the fused silica wafer, electron beam metal evaporation, and liftoff processing. The process of photo-patterning includes wafer cleaning, spin coating the photoresist, soft bake of the resist, ultraviolet light exposure through a chromium mask printed on a 4″×4″ glass plate, resist development, and hard bake of the resist. Following the photo patterning process, a 100-nm-gold layer is deposited on the substrate using electron beam evaporation. A 10-nm layer of chromium is used to enhance the adhesion of gold to the glass wafer; otherwise the gold film gets peeled off easily. gold was chosen as the electrode due to its resistance to corrosion and its inert nature. The width of the electrodes was 20 μm and spacing between the two electrodes was 20 μm.

The microfluidic channel and the mixer chip itself was fabricated in PDMS (Poly-dimethylsiloxane) by using soft lithography. A layer of SU-8 was patterned onto a 3″ Silicon wafer that acts as a master mold. The SU-8 photo-patterning process involves standard cleaning, spin coating, soft baking, exposure, development, and hard baking. After the master mold was fabricated, PDMS (10:1 prepolymer/curing agent) was poured onto the master mold and baked at 80° C. over 2 h for curing. The PDMS channel was then peeled off from the mold. A 5-mm hole and a 3-mm hole were then punched to form the inlet and outlet, respectively. The PDMS substrate was then aligned and bonded to the electrode chip after both substrates have undergone oxygen plasma treatment. The bonded chip was then baked at 70° C. for 40 min to form the irreversible bond. The microfluidic channel had a width of 20 μm and height of 15 um.

### Experimental Setup for Impedance Based Detection

The gold electrodes from the microfluidic chip are connected to a commercial benchtop Impedance Spectroscope (Zurich Instruments, HF2IS). The microfluidic chip is placed inside a Faraday cage to minimize noise and interference. An excitation voltage is applied across the electrodes at a programmable frequency by connecting one electrode to the impedance spectroscope directly whereas the other electrode is connected to a transimpedance amplifier (Zurich Instruments, HF2TA) with a programmable transimpedance gain. The output of the transimpedance amplifier is fed back into the impedance spectroscope for demodulating and filtering the signal. The parameters of the impedance spectroscope can be programmed and for these experiments, an excitation voltage of 5V at a frequency of 5 MHz with a transimpedance gain of 1 kohm and a bandwidth (low pass filter cut off frequency) of 100 Hz was used. The signals are stored on a PC and are processed using MATLAB.

### DNA Nanoball Detection Algorithm

The data obtained from the measurements is processed using an algorithm implemented using MATLAB. The data obtained from the impedance spectroscope typically has a baseline voltage and drift associated. Firstly, the baseline of the signal is computed using a moving average filter. This baseline signal is then subtracted from the original signal to have a normalized signal with only the peaks/spikes present. These peaks represent a change in impedance for a very short duration due to a nanoball or a bead passing through the microfluidic chip across the electrodes. This signal is passed through a filter to remove the background noise and a threshold is applied for the detection of peaks in the response. This threshold is kept 2 μV above the noise. The noise of the signal is computed using the variation of the response in the control group when there are no peaks in the response. The outliers are then removed from the peaks and the number of peaks detected are noted as the number of nanoballs detected by the system.

### Optimization of Electrical Parameters of the Impedance Spectroscope

Multiple configurations of the electrical parameters of the impedance spectroscope were tested for optimizing the detection of the DNA nanoballs. Experiments were performed by passing Dynabeads™ MyOne™ in 1× Phosphate Buffered Saline solution (PBS) for 10 minutes and then recording the response. The data was analyzed for baseline voltage, the signal-to-noise ratio, the number of beads detected, and the peak amplitude. All the measurements were done in the Faraday cage to reduce noise and interference from external sources. The electrical parameters were changed in a progressive fashion. First, the excitation voltage was increased. The increase in excitation voltage results in a better signal. However, the electrodes break down at very high voltage and therefore the voltage was progressively increased until it reached the maximum allowable limit. The transimpedance gain, the bandwidth, and the excitation frequency were also changed to find the optimal electrical parameters for the detection of 1 μm particles in the microfluidic chip. Although the parameters were optimized for the detection of 1 μm beads, experiments showed that there was a similar improvement for detection of the DNA nanoballs.

### SARS-COV-2 Clinical Samples

Anonymized or pseudo anonymized surplus aliquots from 30 SARS-COV-2 positive and 10 negative nasopharyngeal samples that had previously been clinically diagnosed for COVID-19 by RT-PCR were obtained in early February 2022 by demand of the Public Health Agency of Sweden. Specimens, originating from central Sweden, were collected in a fixed volume of 1 mL physiological saline (0.9% NaCl) and inactivated by heat (70° C. for 50 min) upon arrival to the laboratory, and subsequently subjected to extraction-free SARS-CoV-2 RT-PCR. Samples were stored at 4° C. prior to use. The SARS-COV-2 RT-PCR assay was an improved multiplex version of the extraction-free protocol developed by Smyrlaki et. al. (46) with increased sample input and reaction volume and increased sensitivity (47). For each reaction, 24 uL RT-PCR master mix was prepared, containing 7.5 uL TaqPath 1-Step RT-qPCR Master Mix, CG (ThermoFisher, containing ROX as passive reference), 0.9 uL 10% Tween20 (Sigma), N1 primer-probe mix (forward: GACCCCAAAATCAGCGAAAT; SEQ ID NO:60, reverse: TCTGGTTACTGCCAGTTGAATCTG; SEQ ID NO:61, probe: FAM-ACCCCGCATTACGTTTGGTGGACC-BHQ1; SEQ ID NO:62, Integrated DNA Technologies), S primer-probe mix (forward: ATATTCTAAGCACACGCCTATTATAG; SEQ ID NO:63, reverse: CTACCAATGGTTCTAAAGCCGAA; SEQ ID NO:64, probe: Cy5-GAGCCAGAAGATCTCCCTCAGGGT-BXQ2; SEQ ID NO:65, Merck), RNaseP primer-probe mix (forward: AGATTTGGACCTGCGAGCG; SEQ ID NO:66, reverse: GAGCGGCTGTCTCCACAAGT; SEQ ID NO:67, probe: HEX-TTCTGACCTGAAGGCTCTGCGCG-BHQ1; SEQ ID NO:68, Merck; detecting the Omicron B A.1 sub lineage), and nuclease free water up to 24 uL. Primer/Probe concentrations in the final reactions were 246/62 nM (N1), 491/125 nM(S), and 122/37 nM (RNaseP). For RT-PCR testing, 6 uL heat-inactivated nasopharyngeal swab sample (in 0.9% NaCl) was added to optical 96-well PCR plates (EnduraPlate, Applied Biosystems) containing 24 uL master mix. RT-PCR was performed on QuantStudio real-time PCR machines (Applied Biosystems) using the QuantStudio Design &amp; Analysis Software v1.5.2 and temperature cycles: 25° C. for 2 min, 50° C. for 10 min, 95° C. for 2 min, and 40 cycles of 95° C. for 3 s and 56° C. for 30 s. Informed consent for the use of anonymized/pseudo anonymized surplus aliquots obtained in routine clinical diagnostics was not obtained and not required, which is in accordance with the study permit obtained by the Swedish Ethical Review Authority (Dnr 2020-01945 and 2022-01139-02, Etikprövningsmyndigheten). All SARS-COV-2 positive samples (n=30) used in our study had their viral genome sequenced using the Illumina COVIDSeq Test kit (Illumina).

### Volunteer Saliva Experimental Set-Up

Saliva samples were obtained from healthy anonymized volunteers which were directly stored at 4 degrees. To evaluate whether the modified RT-LAMP could produce DNA Nanoballs in human saliva different volumes (1.5 ul or 5 ul) of saliva were first mixed with our synthetic fragments of SARS-COV-2-RNA (10\hat{ }5, 10\hat{ }5 or zero-water control). Subsequently, it was tested whether heating or not this mixture at 95 degrees for 15 min prior to adding to the standard RT-LAMP mastermix in 20 ul final reaction volume impacted later DNA Nanoball production

TABLE 1

| Name of primer | Sequence (5′-3′) |
|---|---|
| As1 F3 FAM | /5-6-FAM/CGGTGGACAAATTGTCAC (SEQ ID NO: 1) |
| As1 LF | TTACAAGCTTAAAGAATGTCTGAACACT (SEQ ID |
| | NO: 2) |
| As1 LB | TTGAATTTAGGTGAAACATTTGTCACG (SEQ ID NO: 3) |
| As1 B3 | CTTCTCTGGATTTAACACACTT (SEQ ID NO: 4) |
| As1e FIP | TCAGCACACAAAGCCAAAAATTTATTTTTCTGTGCAA |
| | AGGAAATTAAGGAG (SEQ ID NO: 5) |
| As1e BIP | TATTGGTGGAGCTAAACTTAAAGCCTTTTCTGTACAA |
| | TCCCTTTGAGTG (SEQ ID NO: 6) |
| As1_LF_Two_repeat | TTACAAGCTTAAAGAATGTCTGAACACTAAATTACAA |
| Compaction | GCTTAAAGAATGTCTGAACACT (SEQ ID NO: 7) |
| As1_LB Two_repeat | TTGAATTTAGGTGAAACATTTGTCACGAAATTGAATT |
| Compaction | TAGGTGAAACATTTGTCACG (SEQ ID NO: 8) |
| As1_LF Two_repeat | TTACAAGCTTAAAGAATGTCTGAACACTAAATTACAA |
| Compaction__InvdT | GCTTAAAGAATGTCTGAACACT/3InvdT/(SEQ ID NO: 9) |
| As1_LB Two_repeat | TTGAATTTAGGTGAAACATTTGTCACGAAATTGAATT |
| Compaction__InvdT | TAGGTGAAACATTTGTCACG/3InvdT/(SEQ ID NO: 10) |
| As1_LF Three_repeat | TTACAAGCTTAAAGAATGTCTGAACACTAAATTACAA |
| Compaction_InvdT | GCTTAAAGAATGTC |
| | TGAACACTAAATTACAAGCTTAAAGAATGTCTGAACA |
| | CT/3InvdT/(SEQ ID NO: 11) |
| As1_LB Three_repeat | TTGAATTTAGGTGAAACATTTGTCACGAAATTGAATT |
| Compaction_InvdT | TAGGTGAAACATTT |
| | GTCACGAAATTGAATTTAGGTGAAACATTTGTCACG |
| | /3InvdT/(SEQ ID NO: 12) |

TABLE 2
Asle SARS_CoV_2 RT LAMP and compaction primers

| Name of primer | Sequence (5′-3′) |
|---|---|
| Myco_F3 | CCTATCCGTATGGTGGATAACG (SEQ ID NO: 13) |
| Myco_B3 | GTCGGAAGCTCCTATGACAAT (SEQ ID NO: 14) |
| Myco_FIP | ACCGGATCGATGTGTACTGAGA/TTTT/ |
| | CCAACAAGAAGGCGTACTC (SEQ ID NO: 15) |
| Myco_BIP | ATCCAACCGTCGGTCGGA/TTTT/ |
| | TTGATCGTCTCGGCTAGT (SEQ ID NO: 16) |
| Myco_LF | GATCGAGCAAGCCATCTG (SEQ ID NO: 17) |
| Myco_LB | TGATCAGCTCGGTCTTGTA (SEQ ID NO: 18) |
| Myco_three_repeat | GATCGAGCAAGCCATCTGAAAGATCGAGCAAGCCAT |
| compaction_LF | CTGAAAGATCGAGCAAGCCATCTG (SEQ ID NO: 19) |
| Myco_three_repeat | TGATCAGCTCGGTCTTGTAAAATGATCAGCTCGGTCT |
| compaction LB | TGTAAAATGATCAGCTCGGTCTTGTA (SEQ ID NO: 20) |
| HIV_LF | CTTTCCAGAGAAGCTTTGCT (SEQ ID NO: 21) |
| HIV_LB | AGCAAAGATCATTAGGGATTAT (SEQ ID NO: 22) |
| HIV_FIP | CTTGTATTACTACTGCCCCTTCACGATCCACTTTGGA |
| | AAGGACC (SEQ ID NO: 23) |
| HIV_BIP | TGACATAAAAGTAGTGCCAAGAAGATTTTACAATCA |
| | TCACCTGCCATC (SEQ ID NO: 24) |
| HIV_F3 | GGTTTATTACAGGGACAGCA (SEQ ID NO: 25) |
| HIV_B3 | ATCCTGTCTACTTGCCAC (SEQ ID NO: 26) |
| HIV_three_repeat | CTTTCCAGAGAAGCTTTGCTAAACTTTCCAGAGAAG |
| Compaction LF | CTTTGCTAAACTTTCCAGAGAAGCTTTGCTA (SEQ ID |
| | NO: 27) |
| HIV_three_repeat | AGCAAAGATCATTAGGGATTATAAAAGCAAAGATCA |
| Compaction_LB | TTAGGGATTATAAAAGCAAAGATCATTAGGGATTAT |
| | (SEQ ID NO: 28) |
| Influenza LF | ACTTGTCTTGGGGAATATCTC (SEQ ID NO: 29) |
| Influenza BIP | ATGCTGGAGCAAAAAGCT (SEQ ID NO: 30) |
| Influenza FIP | CGAGTCATGATTGGGCCATGACAGTGTCATCATTTG |
| | AAAGGTTT (SEQ ID NO: 31) |
| Influenza_BIP | AAGGTGTAACGGCAGCATGTCCGAATTTCCTTTTTTA |
| | ACTAGCCAT (SEQ ID NO: 32) |
| Influenza_F3 | GCTAAGAGAGCAATTGAGC (SEQ ID NO: 33) |
| Influenza_B3 | ATGTAGGATTTGCTGAGCT (SEQ ID NO: 34) |
| Influenza_three | ACTTGTCTTGGGGAATATCTCAAAACTTGTCTTGGGG |
| repeat | AATATCTCAAAACTTGTCTTGGGGAATATCTC |
| compaction__LF | (SEQ ID NO: 35) |
| Influenza_three | ATGCTGGAGCAAAAAGCTAAAATGCTGGAGCAAAA |
| repeat | AGCTAAAATGCTGGAGCAAAAAGCT (SEQ ID NO: 36) |
| compaction_LF | |
| Beta_Lac_F3 | TTGGAATGCTCACTTTACTGAA (SEQ ID NO: 37) |
| Beta_Lac_B3 | CGATATCGCGCGTCTGTC (SEQ ID NO: 38) |
| Beta_Lac_FIP | TGCTTGGTTCGCCCGTTTAAGATGGCGTAGTTGTGCT |
| | CTGG (SEQ ID NO: 39) |
| Beta_Lac_BIP | TTCCCAATAGCTTGATCGCCCTCCCACTTAAAGACTT |
| | GGTGT (SEQ ID NO: 40) |
| Beta_Lac_LF | ATTGGTAAATCCTTGCTGCT (SEQ ID NO: 41) |
| Beta_Lac_LB | TGGGCGTGGTTAAGGATGA (SEQ ID NO: 42) |
| Beta_Lac_three_ | ATTGGTAAATCCTTGCTGCTAAAATTGGTAAATCCTT |
| repeat | GCTGCTAAAATTGGTAAATCCTTGCTGCT (SEQ ID |
| compaction_LF | NO: 43) |
| Beta_Lac_three | TGGGCGTGGTTAAGGATGAAAATGGGCGTGGTTAAG |
| repeat | GATGAAAATGGGCGTGGTTAAGGATGA (SEQ ID |
| compaction_LB | NO: 44) |

TABLE 3
Other targets RT LAMP and compaction primers

| Primer name | Sequence (5'-3′) |
|---|---|
| Ultramer_1__ | ACCTCCATGGTCCTCGACGCGATCGAGCAAGCCATCTGGACCC |
| myco | GCCAACAAGAAGGCGTACTCGACCTGAAAGACGTTATCCACC |
| | ATACGGATAGGGGATCTCAGTACACATCGATCCGGTTCAGCGA |
| | GCGGCTCGCCGAGGCAGGCATC (SEQ ID NO: 45) |
| Ultramer_2_ | CCGCCAGGGCTTGCCGGGTTTGATCAGCTCGGTCTTGTATAGG |
| myco | CCGTTGATCGTCTCGGCTAGTGCATTGTCATAGGAGCTTCCGA |
| | CCGCTCCGACCGACGGTTGGATGCCTGCCTCGGCGAGCCGCTC |
| | GCTGAACCGGATCGATGTGTA (SEQ ID NO: 46) |
| For_T7_Myco | ATCGATTAATACGACTCACTATAGGGACCTCCATGGTCCTCGA |
| | CGC (SEQ ID NO: 47) |
| Rev_T3_Myco | TACGTTAATTAACCCTCACTAAAGCCGCCAGGGCTTGCC (SEQ |
| | ID NO: 48) |
| Ultramer_1_ | ACAAAAATTCAAAATTTTCGGGTTTATTACAGGGACAGCAGAA |
| HIV | ATCCACTTTGGAAAGGACCAGCAAAGCTCCTCTGGAAAGGTG |
| | AAGGGGCAGTAGTAATACAAGATAATAGTGACATA (SEQ ID |
| | NO: 49) |
| Ultramer_2_ | CTCATCCTGTCTACTTGCCACACAATCATCACCTGCCATCTGTT |
| HIV | TTCCATAATCCCTAATGATCTTTGCTTTTCTTCTTGGCACTACTT |
| | TTATGTCACTATTATCTTGTATTACTACTGC (SEQ ID NO: 50) |
| For_T7_HIV | ATCGATTAATACGACTCACTATAGGGACAAAAATTCAAAATTT |
| | TCGGGTTTATTA (SEQ ID NO: 51) |
| Rev_T3_HIV | TACGTTAATTAACCCTCACTAAAGCTCATCCTGTCTACTTGCCA |
| | (SEQ ID NO: 52) |
| Ultramer_1 | GATTTCATCGATTATGAGGAGCTAAGAGAGCAATTGAGCTCAG |
| Influenza | TGTCATCATTTGAAAGGTTTGAGATATTCCCCAAGACAAGTTC |
| | ATGGCCCAATCATGACTCGAACAAAGGTGTAACGGCAGCATG |
| | TCCTCATGCTGG (SEQ ID NO: 53) |
| Ultramer_2 | GACTTCTTTCCCTTTATCATTAATGTAGGATTTGCTGAGCTTTG |
| Influenza | GGTATGAATTTCCTTTTTTAACTAGCCATATTAAATTTTTGTAG |
| | AAGCTTTTTGCTCCAGCATGAGGACATGCTGCCGTTACACCTTT |
| | GTTCGAGTCA (SEQ ID NO: 54) |
| For_T7_ | ATCGATTAATACGACTCACTATAGGGGATTTCATCGATTATGA |
| Influenza | GGA (SEQ ID NO: 55) |
| Rev_T3_ | TACGTTAATTAACCCTCACTAAAGGACTTCTTTCCCTTTATCAT |
| Influenza | (SEQ ID NO: 56) |
| Ultramer_1 | ACCTCCATGGTCCTCGACGCTTGGAATGCTCACTTTACTGAAC |
| Beta_Lac | ATAAATCACAGGGCGTAGTTGTGCTCTGGAATGAGAATAAGC |
| | AGCAAGGATTTACCAATAATCTTAAACGGGCGAACCAAGCATT |
| | TTTACCCGCATCTACCTTTAAAA (SEQ ID NO: 57) |
| Ultramer_2 | ATGATCGCGATTCCAAGTGGCGATATCGCGCGTCTGTCCATCC |
| Beta_Lac | CACTTAAAGACTTGGTGTTCATCCTTAACCACGCCCAAATCGA |
| | GGGCGATCAAGCTATTGGGAATTTTAAAGGTAGATGCGGGTA |
| | AAAATGCTTGGTTCGCCCGTTT (SEQ ID NO: 58) |
| For_T7_Beta_ | ATCGATTAATACGACTCACTATAGGGACCTCCATGGTCCTCGA |
| Lac | CGC (SEQ ID NO: 47) |
| Rev_T3_Beta | TACGTTAATTAACCCTCACTAAAGATGATCGCGATTCCAAGTG |
| Lac | GCGATA (SEQ ID NO: 59) |

### Results

### Electric Detection of Self-Assembled DNA Nanoballs

A one pot reaction was developed that does not rely on external beads. During a standard RT-LAMP amplification a series of concatemer products of different lengths are generated. Oligonucleotides complementary to a common region present in the amplicons can be used to “staple” them together into a DNA nanoball.

The use of compaction oligos could enable the generation of the DNA nanoballs in parallel to the isothermal DNA amplification (FIG. 1A). To test this novel concept it was applied to the detection of SARS-COV-2 RNA. The standard Asle (22) RT-LAMP LF and LB primers were initially modified with two repeat compaction oligos targeting the SARS-CoV-2 genome in a simplified reaction using synthetic SARS-COV-2 RNA (See Methods for details). Self-assembling compact DNA Nanoballs were generated ranging from 1 μm to 2 μm which can be seen under fluorescent microscope (FIG. 1B-1C). It was then validated if it was possible to detect these DNA Nanoballs via impedance detection in the microfluidic device as illustrated in (FIG. 1D-1E).

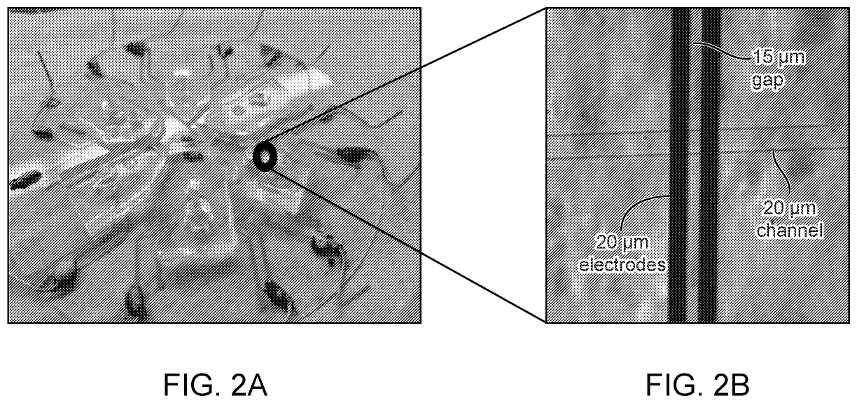

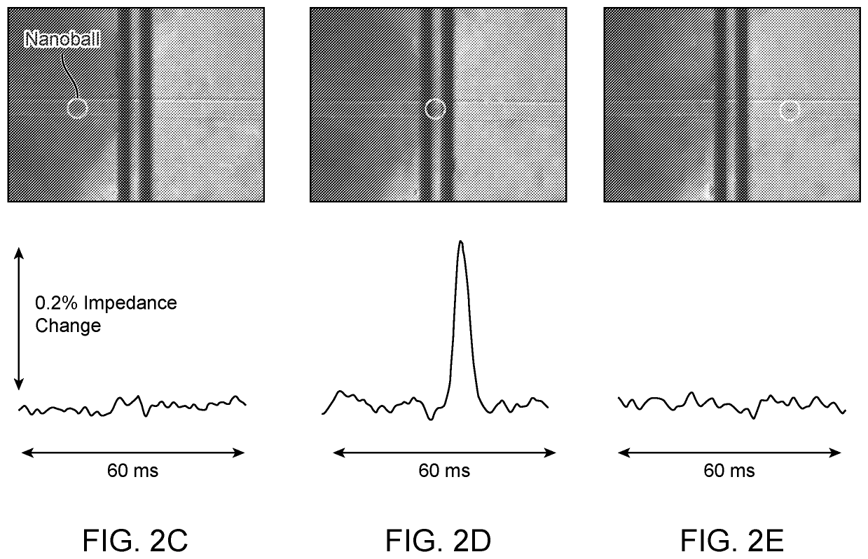

A simple microfluidic chip made of PDMS on a glass substrate with gold electrodes was used (a photograph and microscopic image can be seen in FIG. 2A and FIG. 2B respectively). The microfluidic chip uses passive flow due to capillary action thereby eliminating the need of tedious microfluidic tubes and pumps which add to the complexity of the detection mechanism. This simple passive flow of the DNA nanoballs is depicted in FIG. 1D where the nanoballs flow passively through the channel above the gold electrodes. These gold electrodes are connected to an electronic readout system depicted in FIG. 1E. As the nanoball passes through the gold electrodes, they occlude the current path and the electric field between the two gold electrodes. This results in a change in impedance which results in a peak signature at the output of the readout system as seen in FIG. 2C-2E as the nanoball is passing through the sensing region between the two electrodes.

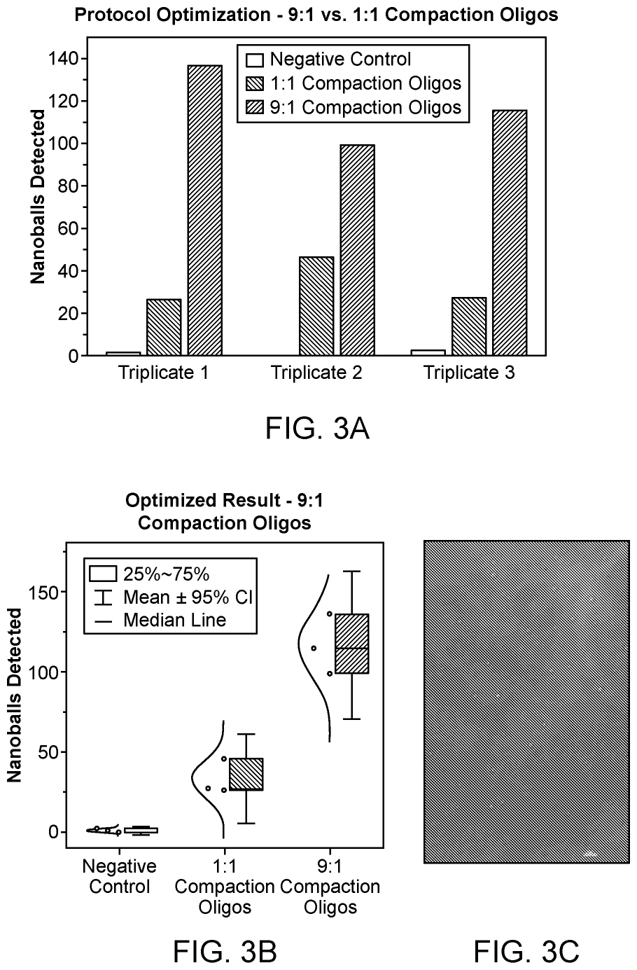

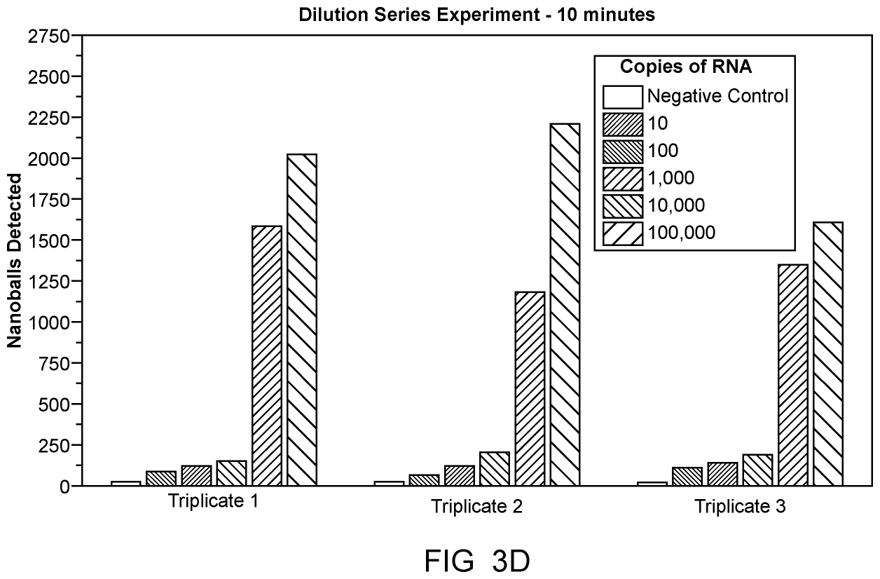

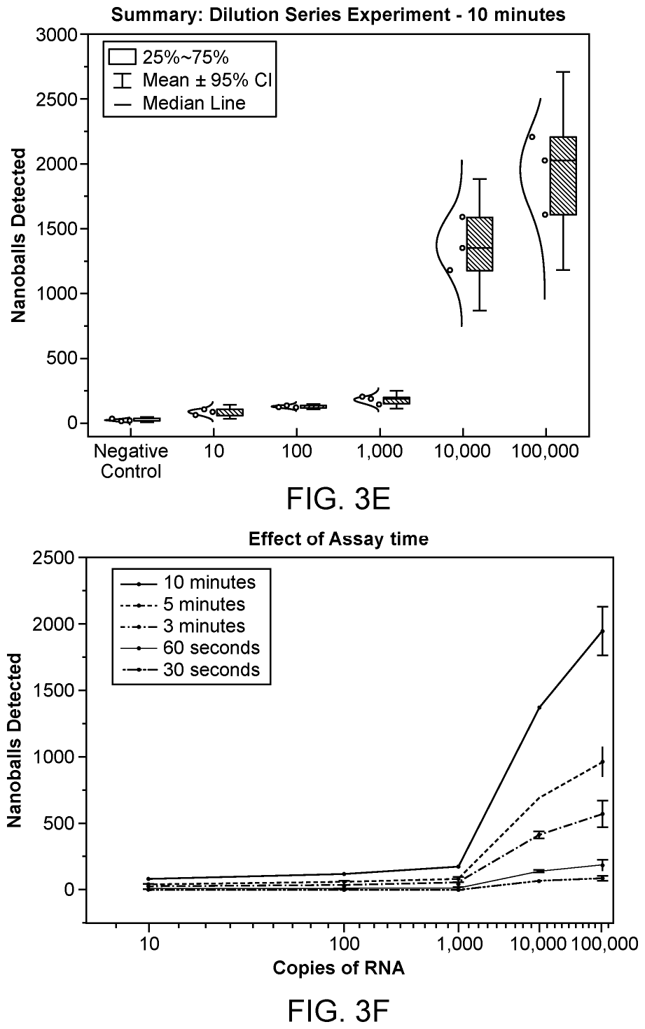

These initial RT-LAMP compaction experiments revealed the formation of numerous, distinct and regular ball-like structures comparable in size to 1 μm beads as shown in FIG. 3C. Next varying the concentration of compaction oligos (either 1:1 ratio or 9:1 ratio of compaction vs amplification oligos, see methods for details) was experimented with. These experiments were repeated in triplicates along with negative water control RT-LAMP reactions as shown in FIG. 3A. As seen in FIG. 3B, the use of a higher proportion of compaction oligos (9:1) yielded a higher average number of detected DNA nanoballs (>100), and therefore this compaction oligo ratio was subsequently used for the following experiments.

### Optimization of Impedance Detection of DNA Nanoballs

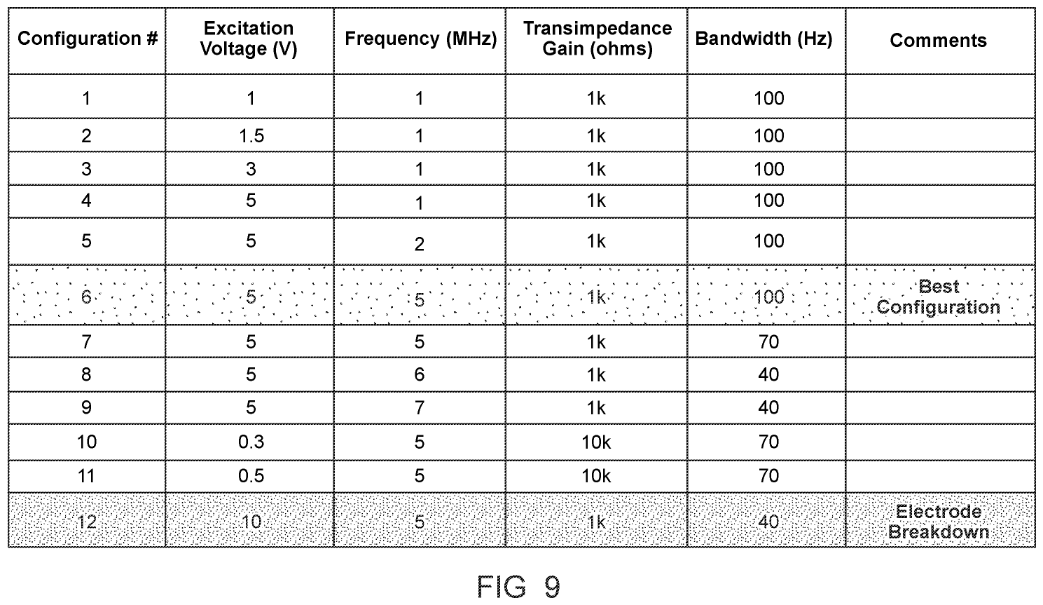

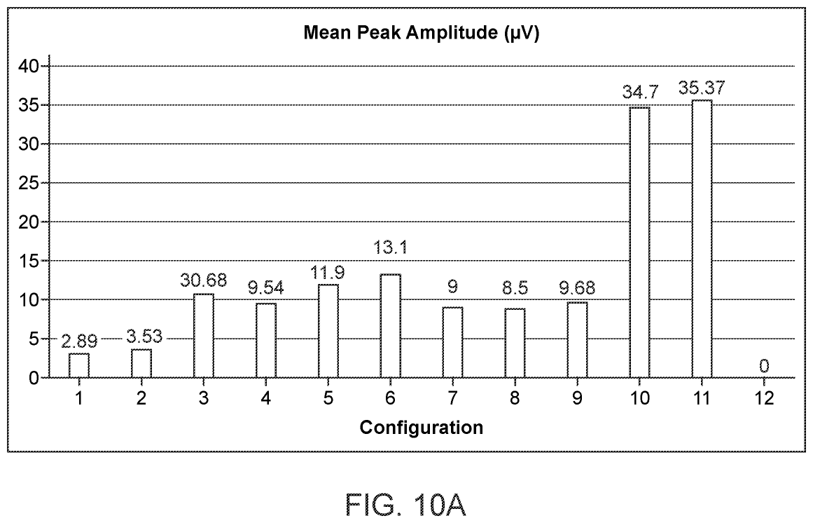

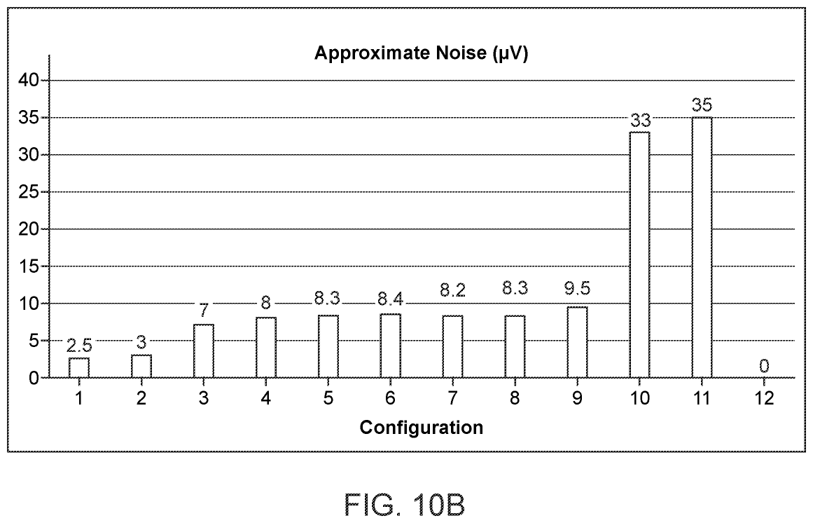

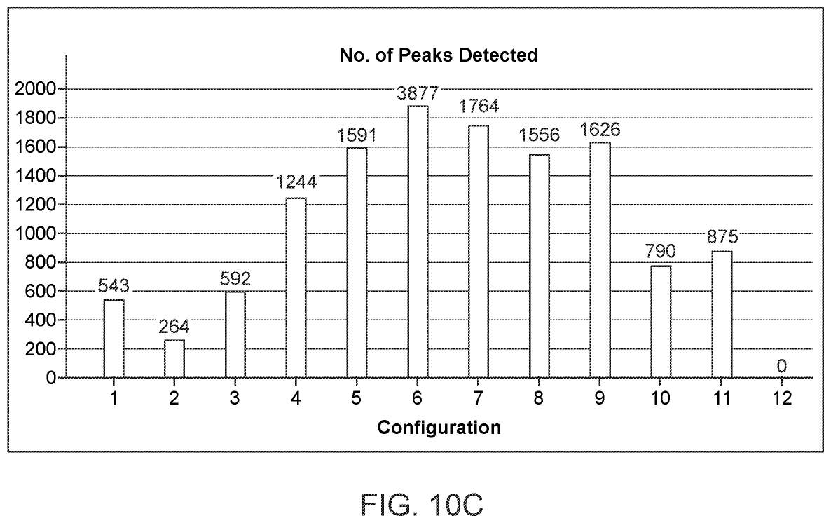

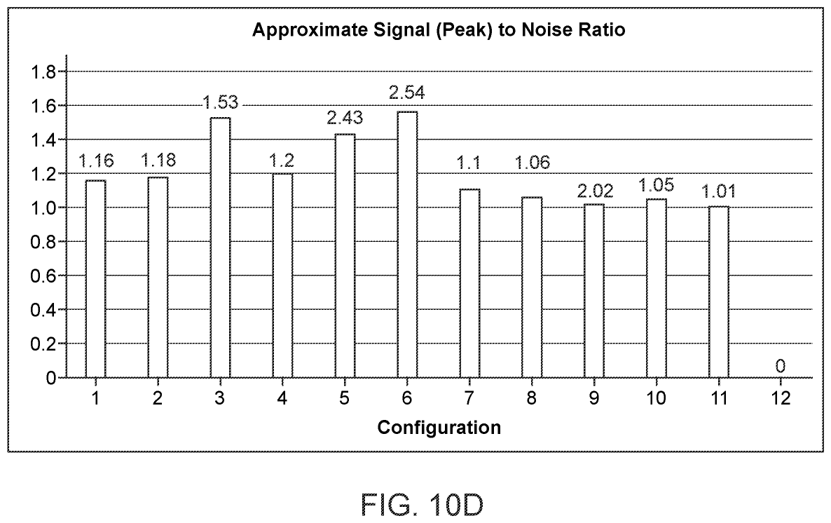

Once the ability to generate DNA nanoballs in a one-pot reaction was validated, the electrical detection of the nanoballs in the microfluidic system was optimized. Different configurations were tested by varying the parameters of the electrical detection system such as excitation voltage, excitation frequency, transimpedance gain, and the Low pass filter bandwidth. These configurations are summarized in FIG. 9 where the detection of 1 μm dynabeads in the microfluidic chip was tested. The following parameters were measured to optimize the sensitivity of the system: number of spikes detected, the baseline voltage, the signal-to-noise ratio, and the spike voltage. It was found out that a high excitation voltage correlates to a higher spike voltage and higher number of spikes detected for the same concentration of Dynabeads. However, too high of a voltage can damage the electrodes and leads to hydrolysis and consequently electrical breakdown. Therefore, 5V was found to be the optimal excitation voltage based on results shown in FIGS. 10A-10D. It was also found out that increasing the frequency yielded improvement up to 5 MHz. Hence, 5 MHz was used as the excitation frequency. Therefore, by the combination of both these initial optimizations: compaction oligo ratio for the formation of nanoballs, and the optimization of the impedance based microfluidic detection system, a substantial improvement was seen in the number of DNA Nanoballs detected. For equivalent synthetic RNA input (10\hat{ }5) detection went from less than 50 DNA Nanoballs to an average of approx 2000 peaks (See FIG. 3A versus FIG. 3D) where the system can easily detect DNA Nanoballs in the form of quantized units that present themselves as spikes/peaks in the signal.

After these improvements the limit of detection of the device was explored. A dilution series experiment was carried out whereby synthetic RNA copies were added in 10-fold increment lower amounts as template in the RT-LAMP reaction. Next, the impedance microfluidic system was used to detect any nanoballs for a period of 10 minutes. This experiment was conducted in triplicate and the nanoballs detected recorded for each experiment FIG. 3D-3E. It was observed from this data that even the lowest concentration of synthetic RNA tested i.e. 10 copies of RNA is easily distinguishable from the Negative Controls with an assay time of 10 minutes and is statistically different from the negative control group (see Methods section for Statistical Analyses). The effect of changing assay time on the Limit of Detection (LOD) and how quickly the device could detect the nanoballs in the current configuration (2 repeat compaction oligos at 9:1 ratio) was next observed. Data from the 10 minute experiments was analyzed by recording the DNA Nanoball spikes observed during various time interval elapses (5 minutes, 3 minutes, 60 seconds, and then 30 seconds). It was found out that even at 30 seconds, the distribution of the nanoballs detected correlated well to the concentration of the synthetic RNA in the mixture as can be seen in FIG. 3F and exhibits a statistically significant difference between the negative control group.

### Three Repeat Compaction Oligo Design Leads to Enhanced Detection

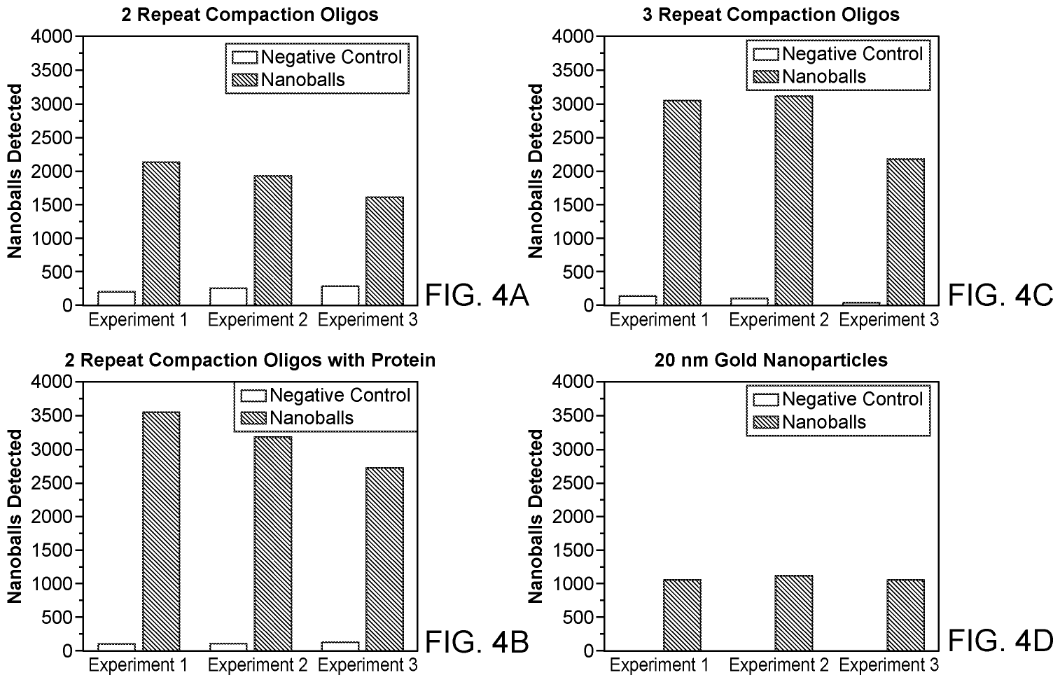

To further improve the ability to detect DNA several strategies were tested. Multiple strategies could lead to improvement of signal to noise ratio, for example increasing the size of the DNA nanoballs or changing their electric impedance by modifying its composition. To increase DNA nanoball size, an additional copy of the common sequence may enhance the ability of the LAMP amplicons to compact and form nanoballs, termed three repeat compaction oligos. In parallel, in efforts to change the electric properties of the generated DNA nanoballs a DNA binding protein (EcoSSB) was added post RT-LAMP reaction. This protein would bind preferentially to the DNA Nanoballs coating them with a protein part. These optimisation trials are seen in FIGS. 4A-4D. The addition of a third compaction oligo repeat sequence in a standard 9:1 ratio substantially increased the number of average DNA Nanoballs detected from approximately 2000 when using two repeat compaction oligos (FIG. 4A) to close to 3000 (FIG. 4B). The addition of a DNA binding protein was also notably able to increase the number of DNA Nanoballs detected in a similar manner and range FIG. 4C. It was also experimented with (FIG. 4D) adding 20 nm streptavidin coated gold beads during the RT-LAMP reaction together with using biotinylated LAMP oligonucleotide. DNA Nanoballs coated with gold may further impact impedance however the results (FIG. 4D) did not lead to an improvement in detection (1000 peaks detected in FIG. 4D) compared to three repeat compaction oligos alone (3000 peaks detected in FIG. 4B). Only the three-repeat compaction oligos were proceeded with since they provided a significant increase in signal without additional handling steps or an increase in overall cost.

### Testing the Production of DNA Nanoballs in Volunteer Human Saliva

In an attempt to test the method and device in a more real world setting human volunteer saliva samples were used. After taking two saliva samples from healthy volunteers the saliva was spiked with various concentrations of synthetic SARS-COV-2 RNA material RT-LAMP reactions were conducted with three repeat compaction oligos. Various parameter changes were tested, including heating the saliva prior to running RT-LAMP and varying the amount of saliva added to the reaction. Approx 1000 DNA Nanoballs could be successfully detected from 10\hat{ }5 RNA spiked in human saliva samples. It was found that lower amounts (1 ul) of Saliva was beneficial compared to higher (5 ul) while prior heating at 95 degrees for 15 min prior to adding to and running RT-LAMP did not impact the number of detected DNA Nanoballs (results not shown).

### Impedance-Based Detection of SARS-COV-2 in Real Samples

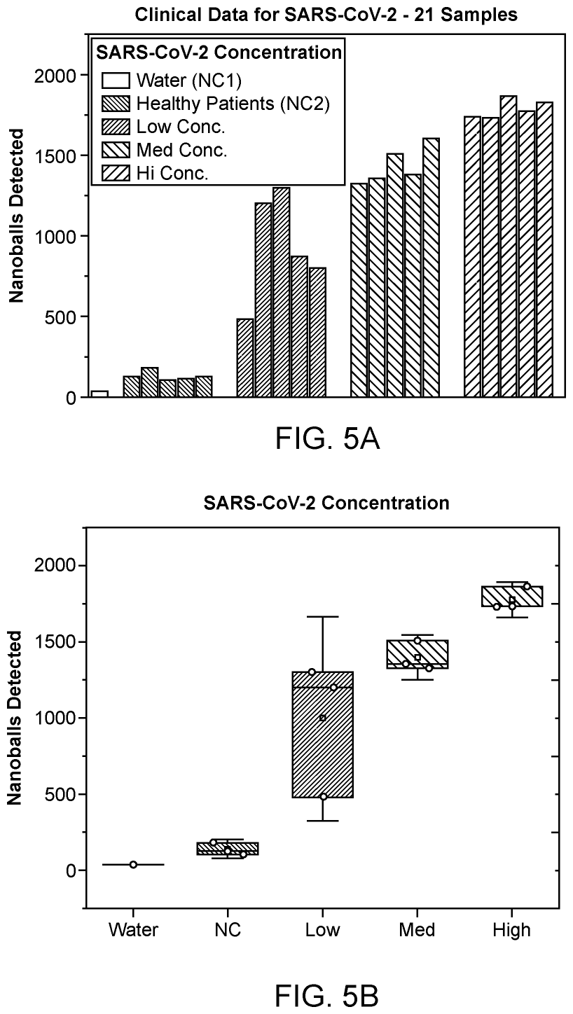

To show the applicability of the approach it was next applied to detect SARS-COV-2 from nasopharyngeal samples sourced from clinical diagnostic testing. Surplus aliquots of heat inactivated nasopharyngeal samples were obtained, which included samples clinically diagnosed for COVID-19 by RT-PCR and negative samples (See Methods and Table XY). Five samples were randomly selected across each of the three different CT ranges: High CT (26-27), Medium CT (22-24) and low CT (17-20) and 5 samples which were tested negative for COVID-19 via RT-PCR were taken. The modified RT-LAMP was run directly on these samples together with the results (FIGS. 5A-5B) demonstrating the ability of the impedance based detection to detect clinically relevant viral levels in non extracted heat inactivated samples. In agreement with the earlier optimization trials in which synthetic SARS-COV-2 RNA was spiked in a strong positive relationship between increased viral load and number of DNA Nanoballs is shown. Here, between 1000-2000 DNA Nanoballs were detected across the range of CT values (27-17). Of further note is the low levels of impedance peaks detected from SARS-COV-2 negative patients despite the complex background material provided in these samples.

### Flexible Detection of DNA Nanoballs from Multiple Pathogens

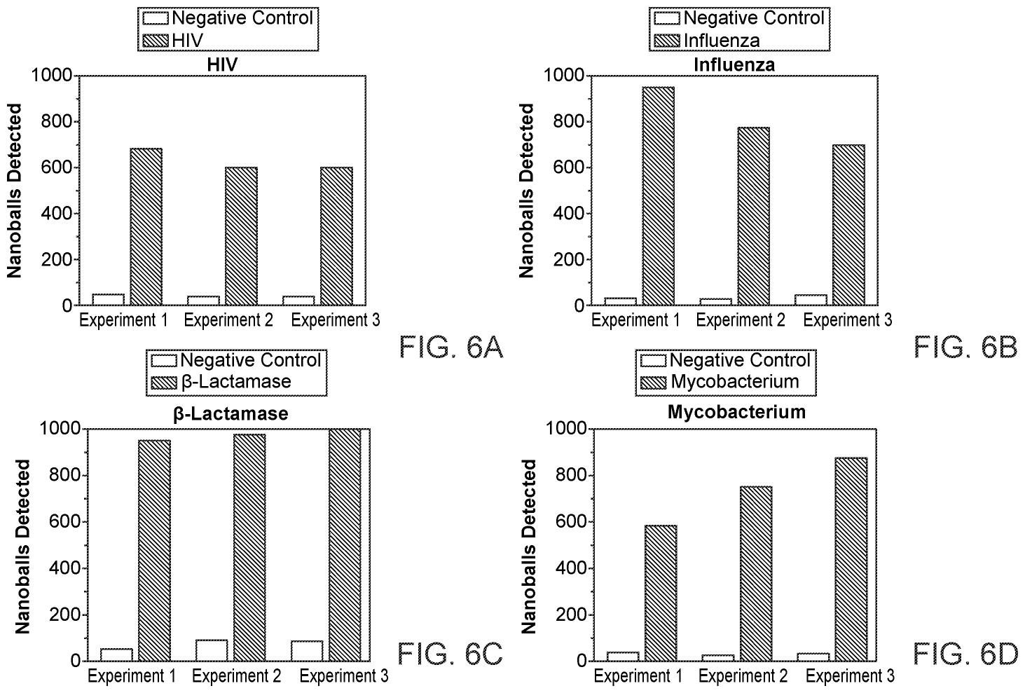

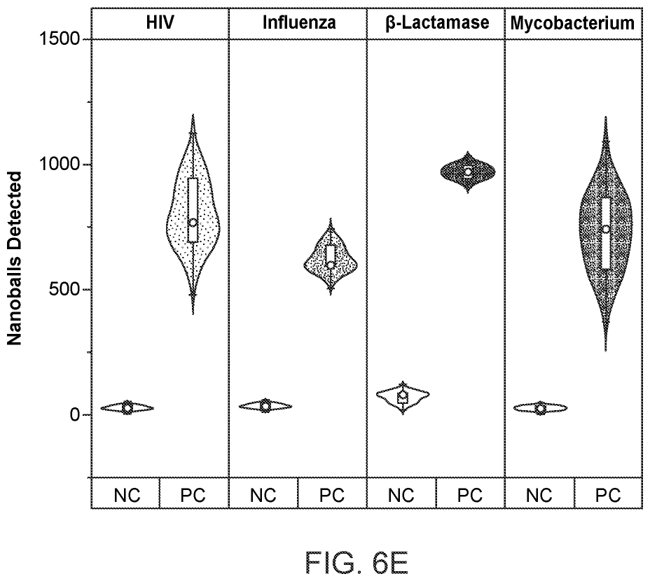

To demonstrate the flexibility and ease of the approach in adapting to novel targets, compaction oligos were designed against multiple pathogens from published RT-LAMP oligos. Three-repeat compaction oligos were designed against relevant diagnostic targets which have in the past and/or continue to pose a threat to public health especially in low income regions. These included oligos against Mycobacterium tuberculosis (39), HIV (40), Influenza-A/H1N1 (41), and a common B-lactamase producing antimicrobial resistance gene (42). As shown in FIGS. 6A-6E, DNA Nanoballs were successfully detected against synthetic genetic target material against HIV, Influenza, Mycobacterium, and β-lactamase. With all targets shown close to 1000 peaks detected and significantly different from water controls.

### Discussion

Here is presented a novel modified RT-LAMP reaction which with the help of compaction oligos produces self-assembling DNA nanoballs which are accurately detected using impedance based electrical detection providing label free-quantized DNA measurements. This approach can be applied for the detection of DNA or RNA and has potential application in multiple fields including forensics, food science, for patient biomarker identification and infection diagnostics. Here is performed a proof of concept focusing on the application of this technology to the detection of pathogens. The straightforward isothermal RT-LAMP reaction works directly on heat inactivated non extracted samples and requires only the addition of two extra compaction oligos to be incorporated into established RT-LAMP mastermix reactions (19) In contrast to protein based detection such as rapid antigen tests which rely on antibody production (5) the nucleic acid based approach allows for greater flexibility and faster adjustment to novel targets. Since the COVID-19 pandemic there has been a huge demand for nucleic acid based detection methods including RT-qPCR and isothermal methods such as RT-LAMP. These often rely on fluorescence or colorimetric indicators (12,24), which increase cost, can lead to false positives and limit scalability in POC settings (25-27). The impedance-based detection of DNA removes the need for beads (35) and enables all components of the reaction to be lyophilized in a rapid one pot reaction which would be a potentially important step towards a scalable POC detection system (45).

The impedance-based detection of the DNA nanoballs offers a quantized measurement of the target DNA or RNA sequence in a specimen. Electrical based detection of DNA has been previously explored but with the aid of microbeads conjugated to DNA (35) While both robust and effective, the need for conjugation of DNA to beads increases cost and precludes scalability particularly in low resource settings. The electrical characterization of DNA in solution has also been performed using an impedimetric sensor (36). These measurements were however not specific to a certain DNA target sequence. In contrast, DNA Nanoballs are only generated in the presence of specific nucleic acid sequences of interest in a standard RT-LAMP reaction providing a targeted approach with a quantized read-out which has a wide range of applications. The electronic components have the inherent advantage of being easily miniaturized (32) and therefore nanoballs may pave the way for a potential portable diagnostic device for rapid and simplified POC detection of DNA or RNA targets of interest.

The rapid and accurate detection of microbes including Ebola, HIV, Malaria parasite and many others remains a challenge across numerous regions (47). In addition, antimicrobial resistant (AMR) pathogens are a growing threat, currently accounting for over 1 million deaths per year (48,49) and projected to account for up to 10 million deaths by 2050 (50). The need for novel, rapid, accurate and scalable detection devices which can be leveraged in POC settings will be a key public health tool in the coming decades. Demonstrated here is the flexibility and ease of the method to generate DNA Nanoballs to detect pathogenic sequences from DNA and RNA of viral and bacterial origin. Experimentation with various oligo modifications (e.g biotin and gold beads) opens up the possibility to produce distinct DNA Nanoballs with unique impedance signatures to potentially allow for a multiplex-based detection of various microbes in a one pot reaction. To further facilitate a POC type system it would be advantageous and feasible in future to combine in one device both the heated RT-LAMP incubation (e.g via a small battery) and subsequent impedance based detection.

### REFERENCES

1. Chen S C A, Halliday C L, Meyer W. A review of nucleic acid-based diagnostic tests for systemic mycoses with an emphasis on polymerase chain reaction-based assays.
2. Galhano B S P, Ferrari R G, Panzenhagen P, de Jesus A C S, Conte-Junior C A. Antimicrobial Resistance Gene Detection Methods for Bacteria in Animal-Based Foods: A Brief Review of Highlights and Advantages. Microorganisms. 2021 May 1 9 (5).
3. Sanjay S T, Fu G, Dou M, Xu F, Liu R, Qi H, et al. Biomarker detection for disease diagnosis using cost-effective microfluidic platforms. Analyst. 2015 Nov. 11 140 (21): 7062.
4. Vandenberg O, Martiny D, Rochas O, van Belkum A, Kozlakidis Z. Considerations for diagnostic COVID-19 tests. Nature Reviews Microbiology. Nature Research; 2020. p. 1-13.
5. Lambert-Niclot S, Cuffel A, le Pape S, Vauloup-Fellous C, Morand-Joubert L, Roque-Afonso A M, et al. Evaluation of a rapid diagnostic assay for detection of sars-cov-2 antigen in nasopharyngeal swabs. J Clin Microbiol. 2020 Aug. 1 58 (8).
6. Mertens P, de Vos N, Martiny D, Jassoy C, Mirazimi A, Cuypers L, et al. Development and Potential Usefulness of the COVID-19 Ag Respi-Strip Diagnostic Assay in a Pandemic Context. Front Med (Lausanne). 2020 May 8; 7:225.
7. Liu R, Han H, Liu F, Lv Z, Wu K, Liu Y, et al. Positive rate of RT-PCR detection of SARS-COV-2 infection in 4880 cases from one hospital in Wuhan, China, from January to February 2020. Clinica Chimica Acta. 2020 Jun. 1; 505:172-5.
8. Dinnes J, Sharma P, Berhane S, van Wyk S S, Nyaaba N, Domen J, et al. Rapid, point-of-care antigen tests for diagnosis of SARS-COV-2 infection. Cochrane Database Syst Rev. 2022 7: CD013705
9. Antigen-detection in the diagnosis of SARS-COV-2 infection using rapid immunoassays Interim guidance. 2020
10. Shen M, Zhou Y, Ye J, Abdullah A L-maskri A A, Kang Y, Zeng S, et al. Recent advances and perspectives of nucleic acid detection for coronavirus. J Pharm Anal. 2020 Apr. 1 10 (2): 97.
11. Esbin M N, Whitney O N, Chong S, Maurer A, Darzacq X, Tjian R. Overcoming the bottleneck to widespread testing: A rapid review of nucleic acid testing approaches for COVID-19 detection. RNA. 2020 Jul. 1 26 (7): 771-83.
12. Moore K J M, Cahill J, Aidelberg G, Aronoff R, Bektaş A, Bezdan D, et al. Loop-Mediated Isothermal Amplification Detection of SARS-COV-2 and Myriad Other Applications. J Biomol Tech. 2021 Sep. 1 32 (3): 228-75.
13. Joung J, Ladha A, Saito M, Segel M, Bruneau R, Huang M li W, et al. Point-of-care testing for COVID-19 using SHERLOCK diagnostics. medRxiv. 2020 May 8 (10).
14. Patchsung M, Jantarug K, Pattama A, Aphicho K, Suraritdechachai S, Meesawat P, et al. Clinical validation of a Cas13-based assay for the detection of SARS-COV-2 RNA. Nature Biomedical Engineering 2020 4:12. 2020 Aug. 26 4 (12): 1140-9.
15. Broughton J P, Deng X, Yu G, Fasching C L, Servellita V, Singh J, et al. CRISPR-Cas12-based detection of SARS-COV-2. Nature Biotechnology 2020 38:7. 2020 Apr. 16 38 (7): 870-4.
16. Becherer L, Borst N, Bakheit M, Frischmann S, Zengerle R, von Stetten F. Loop-mediated isothermal amplification (LAMP)-review and classification of methods for sequence-specific detection. Analytical Methods. 2020 Feb. 13 12 (6): 717-46.
17. Teoh B T, Sam S S, Tan K K, Johari J, Danlami M B, Hooi P S, et al. Detection of dengue viruses using reverse transcription-loop-mediated isothermal amplification. BMC Infect Dis. 2013 Aug. 21 13 (1): 1-9.
18. Yan L, Zhou J, Zheng Y, Gamson A S, Roembke B T, Nakayama S, et al. Isothermal amplified detection of DNA and RNA. Mol Biosyst. 2014 10 (5): 970-1003.
19. Alekseenko A, Barrett D, Pareja-Sanchez Y, Howard R J, Strandback E, Ampah-Korsah H, et al. Direct detection of SARS-COV-2 using non-commercial RT-LAMP reagents on heat-inactivated samples. Sci Rep. 2021 Dec. 1 11 (1).
20. Yu L, Wu S, Hao X, Dong X, Mao L, Pelechano V, et al. Rapid Detection of COVID-19 Coronavirus Using a Reverse Transcriptional Loop-Mediated Isothermal Amplification (RT-LAMP) Diagnostic Platform. Clin Chem. 2020 Jul. 1 66 (7): 975-7.
21. Klein S, Müller T G, Khalid D, Sonntag-Buck V, Heuser A M, Glass B, et al. SARS-CoV-2 RNA Extraction Using Magnetic Beads for Rapid Large-Scale Testing by RT-qPCR and RT-LAMP. Viruses. 2020 Aug. 1 12 (8).
22. Rabe B A, Cepko C. SARS-COV-2 detection using isothermal amplification and a rapid, inexpensive protocol for sample inactivation and purification. Proc Natl Acad Sci USA. 2020 Sep. 29 117 (39): 24450-8.
23. Park G S, Ku K, Baek S H, Kim S J, Kim S il, Kim B T, et al. Development of Reverse Transcription Loop-Mediated Isothermal Amplification Assays Targeting Severe Acute Respiratory Syndrome Coronavirus 2 (SARS-COV-2). J Mol Diagn. 2020 Jun. 1 22 (6): 729-35.
24 Zhang M, Ye J, He J song, Zhang F, Ping J, Qian C, et al. Visual detection for nucleic acid-based techniques as potential on-site detection methods. A review. Anal Chim Acta. 2020 Feb. 22; 1099:1-15.
25. Nagura-Ikeda M, Imai K, Tabata S, Miyoshi K, Murahara N, Mizuno T, et al. Clinical Evaluation of Self-Collected Saliva by Quantitative Reverse Transcription-PCR (RT-qPCR), Direct RT-qPCR, Reverse Transcription-Loop-Mediated Isothermal Amplification, and a Rapid Antigen Test To Diagnose COVID-19. J Clin Microbiol. 2020 Sep. 1 58 (9).
26. DeFina S M, Wang J, Yang L, Zhou H, Adams J, Cushing W, et al. Sali VISION: a rapid saliva-based COVID-19 screening and diagnostic test with high sensitivity and specificity. Scientific Reports 2022 12:1. 2022 Apr. 6 12 (1): 1-14.
27. Yang Q, Meyerson N R, Clark S K, Paige C L, Fattor W T, Gilchrist A R, et al. Saliva twostep for rapid detection of asymptomatic sars-cov-2 carriers. Elife. 2021 Mar. 1; 10.
28. Lisdat F, Schäfer D. The use of electrochemical impedance spectroscopy for biosensing.
29. Lee J A, Hwang S, Kwak J, Park S il, Lee S S, Lee K C. An electrochemical impedance biosensor with aptamer-modified pyrolyzed carbon electrode for label-free protein detection. Sens Actuators B Chem. 2008 Jan. 29; 1 (129): 372-9.
30. Macanovic A, Marquette C, Polychronakos C, Lawrence M F. Impedance-based detection of DNA sequences using a silicon transducer with PNA as the probe layer. Nucleic Acids Res. 2004; 32 (2).
31. Venkatanarayanan A, Keyes T E, Forster R J. Label-free impedance detection of cancer cells. Anal Chem. 2013 Feb. 19; 85 (4): 2216-22.
32. W. KeyesR. Miniaturization of electronics and its limits. IBM J Res Dev. 1988 Jan. 3;
33. Choi S, Goryll M, Yi L, Sin M, Pak ⋅, Wong K, et al. Microfluidic-based biosensors toward point-of-care detection of nucleic acids and proteins.
34. Ghrera A S, Pandey C M, Malhotra B D. Multiwalled carbon nanotube modified microfluidic-based biosensor chip for nucleic acid detection. Sens Actuators B Chem. 2018 Aug. 1; 266:329-36.
35. Sui J, Gandotra N, Xie P, Lin Z, Scharfe C, Javanmard M. Multi-frequency impedance sensing for detection and sizing of DNA fragments. Scientific Reports I. 123AD;11:6490.
36. Liu Y S, Banada P P, Bhattacharya S, Bhunia A K, Bashir R. Electrical characterization of DNA molecules in solution using impedance measurements. Appl Phys Lett. 2008 Apr. 11; 92 (14): 143902.
37. Hong F, Zhang F, Liu Y, Yan H. DNA Origami: Scaffolds for Creating Higher Order Structures. Chem Rev. 2017 Oct. 25 117 (20): 12584-640.
38. Clausson C M, Arngården L, Ishaq O, Klaesson A, Kühnemund M, Grannas K, et al. Compaction of rolling circle amplification products increases signal integrity and signal-to-noise ratio. Scientific Reports 2015 5:1. 2015 Jul. 23; 5 (1): 1-10.
39. Jaroenram W, Kampeera J, Arunrut N, Sirithammajak S, Jaitrong S, Boonnak K, et al. Ultrasensitive detection of Mycobacterium tuberculosis by a rapid and specific probe-triggered one-step, simultaneous DNA hybridization and isothermal amplification combined with a lateral flow dipstick. Scientific Reports 2020 10:1. 2020 Oct. 12 [cited 2022 Sep. 29]; 10 (1): 1-11.
40. Rudolph D L, Sullivan V, Owen S M, Curtis K A. Detection of Acute HIV-1 Infection by RT-LAMP. PLOS One. 2015 May 20 [cited 2022 Sep. 29]; 10 (5).
41. Yu L, Wang J, Li X, Mao L, Sui Y, Chen W, et al. Simultaneous detection of SARS-CoV-2 and pandemic (H1N1) 2009 virus with real-time isothermal platform. Heliyon. 2021 Jul. 1; 7 (7): e07584.
42. Sasano M, Seki M, Takano C, Komine-Aizawa S, Hayakawa S. An improved primer design for the loop-mediated isothermal amplification (LAMP) method to detect oxacillinase (OXA)-48 β-lactamase genes in Gram-negative bacteria for clinical applications. Journal of Infection and Chemotherapy. 2021 Jul. 1; 27 (7): 1005-12.
43. Bruijns B, van Asten A, Tiggelaar R, Gardeniers H. Microfluidic Devices for Forensic DNA Analysis: A Review. [cited 2022 Nov. 22];
44. Huo B, Hu Y, Gao Z, Li G. Recent advances on functional nucleic acid-based biosensors for detection of food contaminants. Talanta. 2021 Jan. 15; 222:121565.
45. Everitt M L, Tillery A, David M G, Singh N, Borison A, White I M. A critical review of point-of-care diagnostic technologies to combat viral pandemics. Anal Chim Acta. 2021 Feb. 15; 1146:184-99.
46. Fiolet T, Kherabi Y, MacDonald C J, Ghosn J, Peiffer-Smadja N. Comparing COVID-19 vaccines for their characteristics, efficacy and effectiveness against SARS-COV-2 and variants of concern: a narrative review. Clinical Microbiology and Infection. 2022 Feb. 1; 28 (2): 202-21.
47. Chen H, Liu K, Li Z, Wang P. Point of care testing for infectious diseases. Clin Chim Acta. 219 June 1493:138-47.
48. Murray C J, Ikuta K S, Sharara F, Swetschinski L, Robles Aguilar G, Gray A, et al. Global burden of bacterial antimicrobial resistance in 2019: a systematic analysis. The Lancet. 2022 Feb. 12 [cited 2022 Apr. 28]; 399 (10325): 629-55.
49. Brown G D, Denning D W, Gow N A R, Levitz S M, Netea M G, White T C. Hidden killers: human fungal infections. Sci Transl Med. 2012 Dec. 19 [cited 2022 Sep. 2]; 4 (165).
50. Dadgostar P. Antimicrobial Resistance: Implications and Costs. Infect Drug Resist. 2019; 12:3903-10.
51. Smyrlaki I, Ekman M, Lentini A, Rufino De Sousa N, Papanicolaou N, Vondracek M, et al. Massive and rapid COVID-19 testing is feasible by extraction-free SARS-COV-2 RT-PCR.
52. Lentini A, Pereira A, Winqvist O, Reinius B. Monitoring of the SARS-COV-2 Omicron B A.1/BA.2 lineage transition in the Swedish population reveals increased viral RNA levels in BA.2 cases. Med (N Y). 2022 September 93 (9): 636-643.e4.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for detecting a target nucleic acid in a sample, comprising:
(a) amplifying the target nucleic acid isothermally in the presence of one or more compaction oligonucleotides to produce a product that comprises condensed amplification products;
(b) flowing the product through a microfluidic channel; and
(c) detecting a change in impedance as the condensed amplification products pass through the microfluidic channel.

2. The method of claim 1, wherein step (a) is done by loop-mediated isothermal amplification (LAMP), reverse transcription loop-mediated isothermal amplification (RT-LAMP) or rolling circle amplification (RCA).

3. The method of claim 1, wherein the sample is a clinical sample obtained from a patient.

4. The method of claim 1, wherein the target nucleic acid is sequence in a viral genome.

5. The method of claim 1, wherein the target sequence is in the SARS-COV-2 genome.

6. The method of claim 1, wherein the microfluidic channel has a width of 10-200 microns.

7. The method of claim 1, wherein the microfluidic channel has two or more electrodes, and wherein the change in impedance is measured when the condensed amplification products pass by the electrodes.

8. The method of claim 1, wherein the channel comprises at least two electrodes that have a width in the range of 1-50 microns, that are spaced apart by 1-50 microns.

9. The method of claim 8, wherein the electrodes provide an alternating current having a frequency of below 100 kHz and a voltage in the range 100-1000 mv.

10. The method of claim 8, wherein the method comprises amplifying a second target nucleic acid isothermally in the presence of one or more compaction oligonucleotides to produce second condensed amplification products, wherein the second condensed amplification products can be distinguished from the product of claim 1 by their impedance profile.

11. A kit comprising:
reagents for amplifying target nucleic acid isothermally;
compaction oligonucleotides for condensing the amplification products;
a microfluidic system comprising;
(i) reaction chamber comprising reagents for amplifying target nucleic acid isothermally and compaction oligonucleotides, for the production of a product that comprises condensed amplification products;
(ii) a microfluidic channel comprising electrodes; and
(iii) an impedance detector, wherein the impedance detector is connected to the electrodes and detects the condensed amplification products passing over the electrodes.

12. The kit of claim 11, wherein the kit further comprises a nasal swab.

13. The kit of claim 11, wherein the reagents and oligonucleotides are in the reaction chamber.

14. A microfluidic system comprising;
(i) reaction chamber comprising reagents for amplifying target nucleic acid isothermally and compaction oligonucleotides, for the production of a product that comprises condensed amplification products;
(ii) a microfluidic channel comprising electrodes; and
(iii) an impedance detector, wherein the impedance detector is connected to the electrodes and detects the condensed amplification products passing over the electrodes.

15. The system of claim 14, wherein the reaction chamber comprising reagents for amplifying the target nucleic acid by LAMP.
